(19) United States
(12) Patent Application Publication
(10) Pub. No.: US 2023/0372554 A1
(43) Pub. Date: Nov. 23, 2023

(54) SIZE CONTROLLED RADIOLABELLED PARTICLES

(71) Applicant: Oncoinvent AS, Oslo (NO)

(72) Inventors: Roy H. Larsen, Oslo (NO); Tina B. Bønsdorff, Oslo (NO)

(21) Appl. No.: 18/044,678

(22) PCT Filed: Sep. 15, 2021
(86) PCT No.: PCT/EP2021/075300

§ 371 Date: Mar. 9, 2023

(30) Foreign Application Priority Data
Sep. 15, 2020 (EP) 20196202.4
Mar. 26, 2021 (EP) 21165246.6

(51) Int. Cl. A61K 51/12; A61K 51/04

(52) U.S. Cl. CPC A61K 51/1251; A61K 51/0489

(57) ABSTRACT

The present disclosure relates to a particle comprising a degradable compound, a radionuclide, and a phosphorus containing additive. Phosphorus containing additives, such as phosphonates, have the unique ability to control the size of particles for medical applications. The applications allow for use of the particles as medicaments and for imaging, especially within the field of cancer.

### FIELD

The present disclosure relates to a particle comprising a degradable compound, a radionuclide, and a phosphorus containing additive. Phosphorus containing additives, such as phosphonates and phosphates, have the unique ability to control the size of particles for medical applications. The applications allow for use of the particles as medicaments and for imaging, especially within the field of cancer.

### BACKGROUND

Radiolabeled microparticles have shown promise for use in radiotherapy of cancer and in treatment of pain as in arthritis treatment with radiosynovectomy (i.e. intra-articular injection of small radioactive particles to treat a synovitis).

It is desirable to use nano- and microparticles which are slowly degradable, and for this purpose crystal particles of non-toxic salts are promising. These crystal particles can be unstable in morphology and size over time, particularly when in suspension. It is important that the particles can be radiolabeled with good yield and that the size distribution and morphology of the crystal particles is stable over time for injections into the body of a patient.

In some applications it may be useful to know particle biodistribution before giving the radiotherapeutic particle suspension. Particles made of stabilized particles radiolabeled with a radionuclide suitable for imaging can be useful for this purpose as it allows the imaging of particle distribution in vivo. Particles of determined size distribution can be useful for imaging purposes to evaluate distribution of fluids in different body compartments, and they can also be applied and designed for specific treatments of certain types of cancer.

A requirement with such particles for medical use relates to controlling the size of the particles and to design them in a way that is optimal for their intended use. The particles can either be non-degradable or degradable. By using non-degradable particles, an advantage can be low risk of systemic toxicity. Disadvantages include potentially more heterogenous radiation dose distribution and some risk of local toxicity from “hot spots”. Stable radiotherapeutic particles have been used for radioembolization using the high energetic beta emitter ⁹⁰Y stably labeled to non-degradable glass spheres (TheraSphere™) or resin based spheres (SIR-Spheres™) for treating primary tumors and metastases to the liver. The liver tissue will in this instance shield against toxic radiation to intestines etc.

By using degradable particles slowly releasing some of the radionuclides, possible advantages includes a more homogenous radiation dose distribution due to improved diffusion of mother nuclides and/or short-lived daughter nuclides and less tendency for “hot spots” causing local toxicity. Possible disadvantages include potential for systemic toxicity due to possible transport of released radionuclide into the blood and further redistribution. Degradable particles are mostly used for other cytotoxic compounds like chemotherapeutics and not for radionuclides at the moment.

A major problem with radiolabelled degradable crystal particles can be the recrystallisation and instability in size of particles when in suspension over time/during storage, the rate of which can be increased for instance by energy input, e.g. by autoclaving. The particles can in various suspensions change size distribution and morphology over time and also when autoclaved in all common solutions tested, including saline, PBS, TRIS.

Thus, an improved delivery system for radionuclides based on particles that are made of non-toxic salts is of interest for medical applications, and further a mode to stabilize the size of degradable particles is warranted.

### SUMMARY

In its broadest aspect, the present invention relates to a particle comprising a degradable compound, a radionuclide, and a phosphorus containing additive.

The degradable compound can be selected from the group consisting of CaCO₃, MgCO₃, SrCO₃, BaCO₃, calcium phosphates including hydroxyapatite Ca₅(PO₄)₃(OH) and fluoroapatite, and composites with any of these as a major constituent. The degradable compound is CaCO₃.

The phosphorus containing additive can be a phosphate selected from the group consisting of orthophosphate, linear oligophosphates and polyphosphates, and cyclic polyphosphates.

The polyphosphate can be selected from the group consisting of pyrophosphate, tripolyphosphate and triphosphono phosphate. The phosphorus containing additive can be a cyclic polyphosphate such as sodium hexametaphosphate (SHMP). The phosphorus containing additive can be a phosphonate. The phosphonate can be a bisphosphonate. The bisphosphonate can be selected from the group consisting of Etidronate, Clodronate, Tiludronate, Pamidronate, Neridronate, Olpadronate, Alendronate, Ibandronate, Risedronate, and Zoledronate. The phosphonate can be a polyphosphonate. The polyphosphonate can be selected from the group consisting of EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid).

The radionuclide can be selected from the group consisting of ²²⁵Ra, ²²⁴Ra, ²²³Ra, ²²⁵Ac, ²²⁷Th, ²¹¹At, ²¹³Bi, ⁶⁴Cu, ⁶⁷Cu, ¹⁶⁶Ho, ¹⁷⁷Lu, ³²P, ¹⁸⁶Re, ¹⁸⁸Re, ¹⁵³Sm, ⁸⁹Sr, ¹⁶¹Tb, ⁹⁰Y ²²⁰Rn, ²¹⁶Po, ²¹²Pb, ²¹²Bi, ²¹²Po, ²⁰⁸Tl, ¹⁸F, ⁶⁷Ga, ^{99m}Tc, ¹¹¹In, ²⁰³Pb, ⁶⁴Cu, ¹⁵²Tb and ¹⁵⁵Tb. The radionuclide can be selected from the group consisting of alpha-radionuclides suitable for therapy consisting of ²²⁵Ac, ²¹¹At, ²¹³Bi, ²¹²Bi, ²²⁵Ra, ²²⁴Ra, ²²³Ra and ²²⁷Th. The radionuclide can be selected from the group consisting of beta-radionuclides suitable for therapy consisting of ⁶⁴Cu, ⁶⁷Cu, ¹⁶⁶Ho, ¹⁷⁷Lu, ³²P, ¹⁸⁶Re, ¹⁸⁸Re, ¹⁵³Sm, ⁸⁹Sr, ¹⁶¹Tb, ⁹⁰Y. The radionuclide can be a beta emitter with alpha-progenies suitable for therapy which is ²¹²Pb. The radionuclide can be selected from the group consisting of alpha-emitting ²²⁴Ra with the progeny radionuclides ²²⁰Rn, ²¹⁶Po, ²¹²Pb, ²¹²Bi, ²¹²Po and ²⁰⁸Tl. The radionuclide can be selected from the group consisting of radionuclide suitable for imaging consisting of ¹⁸F, ⁶⁷Ga, ^{99m}Tc, ¹¹¹In, ²⁰³Pb, ⁶⁴Cu, ¹⁵²Tb and ¹⁵⁵Tb.

The size of the particle can be from 1 nm to 500 μm.

The degradable compound can be selected from the group consisting of PEG modified CaCO₃, protein modified CaCO₃ including mAbs and Fabs, carbohydrate modified CaCO₃, lipid modified CaCO₃, vitamin modified CaCO₃, organic compound modified CaCO₃, polymer modified CaCO₃ and/or inorganic crystal modified CaCO₃.

An aspect of the present invention relates to a composition comprising one or more particles of the present invention.

The composition can be a pharmaceutical composition comprising one or more particles of the present invention, and a diluent, carrier, surfactant, and/or excipient.

The compositions of the present invention can be prepared with an amount of radionuclide that is 1 kBq to 10 GBq per dosing or with an amount of radionuclide that is 50 MBq to 100 GBq suitable for multidose industrial scale production.

The compositions can be a particle suspension comprising monodisperse or polydisperse particles of the present invention.

The compositions of the present invention can be suitable for parenteral use, for instance intravenous, intracavitary and/or intratumor injections.

An aspect of the invention relates to the particle, composition or pharmaceutical composition of the present invention for use as a medicament.

An aspect of the invention relates to the particle, composition or pharmaceutical composition of the present invention for use in intracavitary therapy, radioembolization or radiosynovectomy.

An aspect of the invention relates to the particle, composition or pharmaceutical composition of the present invention for use in the treatment of cancer.

An aspect of the invention relates to the particle, composition or pharmaceutical composition of the present invention for use in the treatment of cancer, wherein the cancer is selected from the group consisting of intraperitoneal cancers, intracranial cancers, pleural cancers, bladder cancers, cardiac cancers, cancers in the subarachnoid cavity, non-cavitary targets such as melanoma, non-small-cell-lung cancer.

An aspect of the invention relates to the particle, composition or pharmaceutical composition of the present invention for use in imaging.

An aspect of the invention relates to the particle, composition or pharmaceutical composition of the present invention for use according to the present invention, which is used in combination with other cancer therapies, such as chemotherapy like taxanes (e.g. paclitaxel, docetaxel), platins (e.g. carboplatin, cisplatin), doxorubicin, mitomycin), DNA repair inhibitors such as PARP inhibitors (e.g. Olaparib, Rucaparib, Niraparib, Talazoparib, Veliparib, Pamiparib, CEP 9722, E7016, and 3-Aminobenzamide), and radioimmunotherapies.

The concentrations of phosphonates and or phosphate compounds are 1 microgram to 1000 milligram per ml, such as 0.1 mg to 10 mg per ml of final solution, or 1 microgram to 1000 milligram per gram particles in the final solution.

An aspect of the invention relates to a method for preparing a particle according to the present invention, the method comprising bringing a degradable compound, a radionuclide, and a phosphorus containing additive in contact with each other with or without using a carrier for the radionuclide. A degradable compound and a radionuclide can form a particle in an initial step, which subsequently is size controlled with the phosphorus containing additive.

### DETAILED DESCRIPTION

The inventors have surprisingly found that application of a phosphorus containing additive can add several beneficial effects in the preparation and maintenance of particles made by degradable compounds and comprising radionuclides. Such compounds are often non-toxic salts, such as crystal particles of the non-toxic salts. The particles can be useful for many types of medical applications including cancer treatment and imaging.

Some of the beneficial effects include stability over time and also at higher temperatures (such as during autoclaving). The stability is enhanced for the maintenance of size distribution, morphology, and/or also without causing release of particle associated radionuclides. Such parameters are very important for the production, shipping and storage of products that are intended for clinical use. It is also important to realize that particles can be designed based on their intended use. It is furthermore possible to design large particles for use in for example radioembolization where they become trapped in blood veins surrounding a tumor due to their size, while for example microparticles can be adapted for cavitary treatment where it is desirable to prevent them from rapidly passing through a membrane (e.g. peritoneum), and nanoparticles are required for intravenous use. It was an unexpected finding that a phosphorus containing additive, such as bisphosphonate, polyphosphonate and polyphosphate, could be used as particle stabilizers thereby maintaining the size distribution and without causing release of particle associated radionuclides. The microparticles kept its size and radiolabel after autoclaving at 120° C. and storing for several days at room temperature, thus allowing sterile production as well as time for shipment and handling of radiolabeled particle suspension for clinical use (see e.g. examples 6, 7, and 11).

Thus, the particles and compositions of the present invention have the beneficial technical advantages of increased stability and prevention of recrystallisation of particles when in suspension over time and/or during storage. The rate of recrystallization and size changes can be increased for instance by energy input, e.g. by autoclaving. The particles will in various suspensions change size distribution and morphology over time, and also when autoclaved in all common solutions tested, including saline, PBS, TRIS. The phosphorus containing additive can therefore be used to increase stability of particles in a composition or suspension. The phosphorus containing additive can therefore be used for prevention of recrystallisation of particles, such as over time and/or with increased energy input, like autoclaving.

Thus, the present invention relates to a particle comprising a degradable compound, a radionuclide, and a phosphorus containing additive. The phosphorus containing additive can be incorporated into the particle, be associated with the surface of the particle or be present in the surroundings of the particle, i.e. in the composition or suspension that the particle is part of. Thus, one aspect of the present invention relates to a composition or suspension comprising a particle, wherein the particle comprises a degradable compound, a radionuclide and a phosphorus containing additive, and wherein the phosphorus containing additive is associated with the particle by being present in the composition or suspension. The presence can be as part of the particle. The presence can be on the surface of the particle. The presence can be in the dispersion of the particle. The presence can be as part of the composition or suspension and/or dispersion of particles. The presence can also be as part of the particle and as part of the composition or suspension of particles. These individual components can be combined into different types of particles that have different characteristics depending on the intended use of the particles.

This means that the phosphorus containing additive, such as EDTMP and/or pamidronate, normally will be found at least in trace amounts on or associated with the individual particles. The phosphorus containing additive, such as EDTMP and/or pamidronate, will therefore normally be at least partly found on or in the particle if particles in a composition, such as a solution, are tested for content of the phosphorus containing additive. The total amount of phosphorus containing additive, such as EDTMP and/or pamidronate, in a composition (such as a solution) will vary depending on the particle design, but will usually mean that at least 0.01-80% of the total amount of phosphorus containing additive in the composition will be in or on the particles, and the remaining part will be in the composition. The at least 0.01-80% of the total amount of phosphorus containing additive in the composition that will be in or on the particles can be 0.1-50%, such as 10-50%, such as 20-80%, such as 10-80%.

### Degradable Compounds

The degradable compound of the present invention can be any compound that can be degraded. The degradation can be done by any route selected from the group consisting of high pH, low pH, temperature, proteases, enzymes, nucleases and/or by cellular processes like endocytosis, which also includes phagocytosis. The degradable compounds can therefore be non-toxic salt or a crystal of a non-toxic salt.

In one or more embodiments of the present invention, the degradable compound can be selected from the group consisting of CaCO₃, MgCO₃, SrCO₃, BaCO₃, calcium phosphates including hydroxyapatite Ca₅(PO₄)₃(OH) and fluoroapatite, and composites with any of these as a major constituent. Major constituent is defined as at least 20% of the total molecular weight of the particle, such as at least 30% of the total molecular weight of the particle, such as at least 40% of the total molecular weight of the particle, such as at least 50% of the total molecular weight of the particle, such as at least 60% of the total molecular weight of the particle, such as at least 70% of the total molecular weight of the particle, such as at least 80% of the total molecular weight of the particle, such as at least 90% of the total molecular weight of the particle, such as at least 95% of the total molecular weight of the particle, such as at least 98% of the total molecular weight of the particle, such as at least 99% of the total molecular weight of the particle.

The degradable compound can be CaCO₃ which is selected from the group consisting of PEG modified CaCO₃, protein modified CaCO₃ including mAbs and Fabs, carbohydrate modified CaCO₃, lipid modified CaCO₃, vitamin modified CaCO₃, organic compound modified CaCO₃, polymer modified CaCO₃ and/or inorganic crystal modified CaCO₃.

The degradable compound can be MgCO₃ which is selected from the group consisting of PEG modified MgCO₃, protein modified MgCO₃ including mAbs and Fabs, carbohydrate modified MgCO₃, lipid modified MgCO₃, vitamin modified MgCO₃, organic compound modified MgCO₃, polymer modified MgCO₃ and/or inorganic crystal modified MgCO₃.

The degradable compound can be SrCO₃ which is selected from the group consisting of PEG modified SrCO₃, protein modified SrCO₃ including mAbs and Fabs, carbohydrate modified SrCO₃, lipid modified SrCO₃, vitamin modified SrCO₃, organic compound modified SrCO₃, polymer modified SrCO₃ and/or inorganic crystal modified SrCO₃.

The degradable compound can be BaCO₃ which is selected from the group consisting of PEG modified BaCO₃, protein modified BaCO₃ including mAbs and Fabs, carbohydrate modified BaCO₃, lipid modified BaCO₃, vitamin modified BaCO₃, organic compound modified BaCO₃, polymer modified BaCO₃ and/or inorganic crystal modified BaCO₃.

The degradable compound can be Ca₅(PO₄)₃(OH) which is selected from the group consisting of PEG modified Ca₅(PO₄)₃(OH), protein modified Ca₅(PO₄)₃(OH) including mAbs and Fabs, carbohydrate modified Ca₅(PO₄)₃(OH), lipid modified Ca₅(PO₄)₃(OH), vitamin modified Ca₅(PO₄)₃(OH), organic compound modified Ca₅(PO₄)₃(OH), polymer modified Ca₅(PO₄)₃(OH) and/or inorganic crystal modified Ca₅(PO₄)₃(OH).

The degradable compound can be fluoroapatite which is selected from the group consisting of PEG modified fluoroapatite, protein modified fluoroapatite including mAbs and Fabs, carbohydrate modified fluoroapatite, lipid modified fluoroapatite, vitamin modified fluoroapatite, organic compound modified fluoroapatite, polymer modified fluoroapatite and/or inorganic crystal modified fluoroapatite.

The composite particles can comprise two or more of these degradable compounds where they combined are a major constituent, as defined above.

The degradable compounds may be used as composites with other salts or proteins or peptides and subject to surface modification by surfactants like oleates and similar.

In a special embodiment, the degradable compounds are used with a compound selected from the group consisting of poly ethylene glycol (PEG) modified particles of the degradable compound or inorganic crystal modified degradable compound.

In a special embodiment the degradable compounds are modified with functional receptor and or antigen binding groups, including monoclonal antibodies and derivatives and vitamins and derivatives allowing receptor or antigen binding of particle to individual target cells and diseased tissues. This means that modifications of the particles relate to the addition of other compounds to degradable compounds. This can be done in various ways, and through interactions such as dipole-dipole interactions, ion-dipole and ion-induced dipole forces, hydrogen bonding, Van der Waals forces, and relative strength of forces.

A chelator can be used, preferentially conjugated to a target affinic molecule, e.g., monoclonal or polyclonal antibody or derivatives of antibody, vitamins or derivatives of vitamins.

Monoclonal antibodies (mAbs), polyclonal antibodies (pAbs), antigen-binding fragments (Fabs) and other types of polypeptides and proteins can be used to include specific targeting in the particle, i.e. by adding a specific targeting molecule, the particles will be able to have enhanced affinity for certain target cells in the body.

### Phosphorus Containing Additive

The phosphorus containing additive can be a phosphate, thus becoming a phosphate containing additive. The phosphorus containing additive can also be a phosphonate, thus becoming a phosphonate containing additive.

Phosphonates and phosphonic acids are organophosphorus compounds containing C—PO(OH)₂ or C—PO(OR)₂ groups (where R=alkyl, aryl). Phosphonic acids, typically handled as salts, are generally non-volatile solids that are poorly soluble in organic solvents, but soluble in water and common alcohols. Thus, the various salts and acids of the phosphonates are also considered parts of the definition of phosphonate.

A phosphoric acid, in the general sense, is a phosphorus oxoacid in which each phosphorus atom is in the oxidation state +5, and is bonded to four oxygen atoms, one of them through a double bond, arranged as the corners of a tetrahedron. Removal of the hydrogen atoms as protons H⁺ turns a phosphoric acid into a phosphate anion. Partial removal yields various hydrogen phosphate anions.

The phosphorus containing additive can be a phosphonate. The phosphonate can be a bisphosphonate. The bisphosphonate can be selected from the group consisting of Etidronate, Clodronate, Tiludronate, Pamidronate, Neridronate, Olpadronate, Alendronate, Ibandronate, Risedronate, and Zoledronate. In one or more embodiments of the present invention the bisphosphonate is Etidronate. In one or more embodiments of the present invention the bisphosphonate is Clodronate. In one or more embodiments of the present invention the bisphosphonate is Tiludronate. In one or more embodiments of the present invention the bisphosphonate is Pamidronate. In one or more embodiments of the present invention the bisphosphonate is Neridronate. In one or more embodiments of the present invention the bisphosphonate is Olpadronate. In one or more embodiments of the present invention the bisphosphonate is Alendronate. In one or more embodiments of the present invention the bisphosphonate is Ibandronate. In one or more embodiments of the present invention the bisphosphonate is Risedronate. In one or more embodiments of the present invention the bisphosphonate is Zoledronate.

The phosphonate can be a polyphosphonate. The polyphosphonate can be selected from the group consisting of EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid). In one or more embodiments of the present invention the phosphonate is EDTMP-ethylenediamine tetra(methylene phosphonic acid). In one or more embodiments of the present invention the phosphonate is DOTMP-1,4,7,10-Tetraazacyclododecane-1,4,7,10-tetrayl-tetrakis(methylphosphonic acid). In one or more embodiments of the present invention the phosphonate is DTPMP-diethylenetriaminepenta(methylene-phosphonic acid).

In one embodiment, the particle of the present invention comprises CaCO₃ as the degradable compound, EDTMP-ethylenediamine tetra(methylene phosphonic acid) as the phosphorus containing additive, and ²²⁴Ra as the radionuclide.

In one embodiment, the particle of the present invention comprises CaCO₃ as the degradable compound, Pamidronate as the phosphorus containing additive, and ²¹²Pb as the radionuclide.

The phosphate containing additives can be selected from the group consisting of orthophosphate, linear oligophosphates and polyphosphates, and cyclic polyphosphates. The polyphosphate can be selected from the group consisting of pyrophosphate, tripolyphosphate and triphosphono phosphate. The phosphorus containing additive can be a cyclic polyphosphate which for example can be sodium hexametaphosphate (SHMP).

The concentrations of phosphonates and or phosphate compounds are 1 microgram to 1000 milligram per ml, such as 0.1 mg to 10 mg per ml of final solution, or 1 microgram to 1000 milligram per gram particles in the final solution. With the particles and methods used in Example 6, where a range from 0.001-0.046 gram EDTMP per gram CaCO₃ was tested, at least 0.013 gram EDTMP per gram CaCO₃ particle was required to obtain size control. For SHMP and pamidronate, all tested concentrations, 0.015-0.200 gram SHMP per gram CaCO₃ and 0.008-0.016 gram pamidronate per gram CaCO₃, resulted in stabilization of size of the polydisperse suspensions. Example 12 (FIG. 5) show that addition of at least 1% EDTMP results in a size control of the microparticles.

Thus, for phosphate containing additives, such as EDTMP, the range for a patient dose can be 1 ug to 1 g per mL or 1 ug to 1 g per g particle, for instance a patient dose could be of 2.5 mg/mL or 25 mg/gram degradable compound, such as CaCO₃. The addition of phosphate containing additives, such as EDTMP, can be 0.1-10%. The addition can also be 0.5-5% or 0.5-2%. The addition can be at least 1%.

The phosphate containing additive, such as EDTMP, can be used in a concentration of 24.4 mg phosphate containing additive, such as EDTMP, for a 10 ml product, with for instance a range of 20 to 30 mg per 10 mL product.

The phosphate containing additive, such as EDTMP, can be used as and an excipient with a final concentration of 5.6 mM (2.44 mg/mL), with a range of 4.6-6.6 mM.

The phosphate containing additive, such as EDTMP, can be used at 24.4 mg/g calcium carbonate, or a range of 10 mg-50 mg per gram calcium carbonate

The phosphate containing additive, such as EDTMP, can be used at 2.4% w/w, with a range of 1-20% w/w.

The amount of phosphate containing additive, such as EDTMP, can be 24.4 mg in 10 ml product.

The phosphate containing additive, such as EDTMP or EDTMPA (acid), can be used as an excipient with a concentration of 5.6 mM (2.44 mg/mL).

The concentration of the phosphate containing additive, such as EDTMP or EDTMPA (acid), can be 5.6 mM±15%.

For phosphonate containing additives, such as Pamidronate, the range for a patient dose can be 1 ug to 1 g per mL or 1 ug to 1 g per g particle, for instance a patient dose could be of 2.5 mg/mL or 25 mg/gram degradable compound, such as CaCO₃. The addition of phosphate containing additives, such as Pamidronate, can be 0.1-10%. The addition can also be 0.5-5% or 0.5-2%. The addition can be at least 1%.

The phosphate containing additive, such as Pamidronate, can be used in a concentration of 10 mg phosphate containing additive, such as Pamidronate, for a 10 ml product, with for instance a range of 5 to 50 mg per 10 mL product.

The phosphate containing additive, such as Pamidronate, can be used as and an excipient with a final concentration of 4 mM (1 mg/mL), with a range of 0.1-10 mM.

The phosphate containing additive, such as Pamidronate, can be used at 0.01 g/g calcium carbonate, or a range of 1 mg-50 mg per gram calcium carbonate

The phosphate containing additive, such as Pamidronate, can be used at 1% w/w, with a range of 0.1-5% w/w.

The amount of phosphate containing additive, such as Pamidronate, can be 10 mg in 10 ml product.

The phosphate containing additive, such as Pamidronate or Pamidronic acid, can be used as an excipient with a concentration of 4 mM (1 mg/mL).

The concentration of the phosphate containing additive, such as Pamidronate or Pamidronate acid can be 4 mM±25%.

Example 13 shows that the sedimentation rate is reduced by reduction of particle size. There are improved features for handling of suspension with advantage in terms of clinical administration of the product. The addition of phosphate containing additives, such as EDTMP, can therefore have and important impact on the sedimentation rate by reduction of particle size. Example 14 shows a comparison of retention of ²¹²Pb and ²²⁴Ra on CaCO₃ microparticles with or without a layer encapsulation, showing how retention of in particular ²¹²Pb is improved by addition of the layer in a product in a phosphate containing additive, such as EDTMP. Example 15 example shows a comparison of the biodistribution of both radium-224 and lead-212 between different amount of layered encapsulated microparticles and free radium-224. Reduced levels of bone uptake with increasing amount of microparticles. Example 16 shows that the cumulative amount of the chemically equivalent stable daughter nuclide ²⁰⁸Pb adsorbed on the MPs increases with time. Example 17 shows known complexation property of EDTMP with both ²¹²Pb and calcium indicates that it is also possible for the ²¹²Pb-EDTMP complex to associate with the MPs.

### Radionuclides

For therapeutic purposes, radionuclides with alpha, beta and Auger electron emissions are considered. Due to variations in mass and energy of the emitted particles, their range in tissue and thereby also their linear energy transfer (LET), which is defined as the energy transferred to matter per unit length, differs significantly. In general, low-LET beta-emitters are believed to be more suitable for treatment of larger tumors than the high-LET alpha- and Auger-emitters, which are preferred for treatment of micrometastases and single cell diseases. The radionuclide in the particles of the present invention can therefore be tailored according to the intended use.

The main medical advantages of alpha particle emitting compounds in local therapy in e.g., the intraperitoneal cavity is the shorter range, typically less than 0.1 mm for alphas compared with mm to cm ranges for beta-particles from medical beta emitters.

Use of alpha-emitters would in an intracavitary setting reduce risk for toxicity due to irradiation of deeper regions of internal organs like the radiosensitive intestinal crypt cells in the case of intraperitoneal (IP) use. Also is the high linear energy transfer of the emitted alpha particles advantageous since very few alpha hits are needed to kill a cell and cellular resistance mechanism like high repair capacity for DNA strand breaks is less of a problem because of the high probability of producing irreparable double strand breaks.

The high effect per decay means less radioactivity is needed reducing the need for shielding of hospital staff and relatives since most alpha- and beta emitters also emits some X-rays and gammas which needs to be shielded against.

In situations where the cancer is characterized as a bulky disease, the longer range of beta particles compared to alpha particles may be advantageous. The longer path length of beta-particles can result in the so-called cross-fire effect, where irradiation of a significantly higher portion of neighboring and distant cells, causing damage to cells that are further away from to the radiolabeled particle occurs.

In the present context, progeny is understood as the radionuclides that are the result of the decay of a parent radionuclide. Thus, when for example ²²⁴Ra is the parent radionuclide, ²²⁰Rn (the daughter radionuclide), ²¹⁶Po (the granddaughter radionuclide), and ²¹²Pb (the great granddaughter radionuclide). ²²⁰Rn, ²¹⁶Po and ²¹²Pb are therefore all considered progeny radionuclides of ²²⁴Ra.

Thus, in one embodiment is the alpha-emitting radionuclide ²²⁴Ra with the daughter radionuclide ²²⁰Rn, the granddaughter radionuclide ²¹⁶Po, and the great granddaughter radionuclide ²¹²Pb. For the particle of the present invention will these all be comprised by the particle when ²²⁴Ra is the radionuclide.

The radionuclide in the particle of the present invention can be selected from the group consisting of ²²⁵Ra, ²²⁴Ra, ²²³Ra, ²²⁵Ac, ²¹²Bi, ²²⁷Th, ²¹¹At, ²¹³Bi, ²¹²Pb, ⁶⁴Cu, ⁶⁷Cu, ¹⁶⁶Ho, ¹⁷⁷Lu ³²P, ¹⁸⁶Re, ¹⁸⁸Re, ¹⁵³Sm, ⁸⁹Sr, ¹⁶¹Tb, ⁹⁰Y ²²⁰Rn, ²¹⁶Po, ²¹²Po, ²⁰⁸Tl, ¹⁸F, ⁶⁷Ga, ⁸⁶Y, ^{99m}Tc, ¹¹¹In, ²⁰³Pb, ⁸³Sr, ¹⁵²Tb and ¹⁵⁵Tb. The radionuclide can be selected from the group consisting of alpha-radionuclides suitable for therapy consisting of ²²⁵Ac, ²¹¹At, ²¹³Bi, ²¹²Bi, ²²⁵Ra, ²²⁴Ra, ²²³Ra and ²²⁷Th. The radionuclide can be selected from the group consisting of beta-radionuclides suitable for therapy consisting of ⁶⁴Cu, ⁶⁷Cu, ¹⁶⁶Ho, ¹⁷⁷Lu ³²P, ¹⁸⁶Re, ¹⁸⁸Re, ¹⁵³Sm, ⁸⁹Sr, ¹⁶¹Tb, ⁹⁰Y. The radionuclide can be a beta emitter with alpha-progenies suitable for therapy which is ²¹²Pb. The radionuclide can be selected from the group consisting of alpha-emitting ²²⁴Ra with the progeny radionuclides ²²⁰Rn, ²¹⁶Po, ²¹²Pb, ²¹²Bi, ²¹²Po and ²⁰⁸Tl. The radionuclide can be selected from the group consisting of radionuclide suitable for imaging consisting of ¹⁸F, ⁶⁷Ga, ⁸⁶Y, ^{99m}Tc, ¹¹¹In, ²⁰³Pb, ⁶⁴Cu, ⁸³Sr, ¹⁵²Tb and ¹⁵⁵Tb. In one or more embodiments of the present invention the radionuclide is ²²⁵Ra. In one or more embodiments of the present invention the radionuclide is ²²⁴Ra. In one or more embodiments of the present invention the radionuclide is ²²³Ra. In one or more embodiments of the present invention the radionuclide is ²²⁵Ac. In one or more embodiments of the present invention the radionuclide is ²¹²Bi. In one or more embodiments of the present invention the radionuclide is ²²⁷Th. In one or more embodiments of the present invention the radionuclide is ²¹¹At. In one or more embodiments of the present invention the radionuclide is ²¹³Bi. In one or more embodiments of the present invention the radionuclide is ²¹²Pb. In one or more embodiments of the present invention the radionuclide is ⁶⁴Cu. In one or more embodiments of the present invention the radionuclide is ⁶⁷Cu. In one or more embodiments of the present invention the radionuclide is ¹⁶⁶Ho. In one or more embodiments of the present invention the radionuclide is ¹⁷⁷Lu. In one or more embodiments of the present invention the radionuclide is ³²P. In one or more embodiments of the present invention the radionuclide is ¹⁸⁶Re. In one or more embodiments of the present invention the radionuclide is ¹⁵³Sm. In one or more embodiments of the present invention the radionuclide is ⁸⁹Sr. In one or more embodiments of the present invention the radionuclide is ¹⁶¹Tb. In one or more embodiments of the present invention the radionuclide is ⁹⁰Y. In one or more embodiments of the present invention the radionuclide is ²²⁰Rn. In one or more embodiments of the present invention the radionuclide is ²¹⁶Po. In one or more embodiments of the present invention the radionuclide is ²¹²Bi. In one or more embodiments of the present invention the radionuclide is ²¹²Po. In one or more embodiments of the present invention the radionuclide is ²⁰⁸Tl. In one or more embodiments of the present invention the radionuclide is ¹⁸F. In one or more embodiments of the present invention the radionuclide is ⁶⁷Ga. In one or more embodiments of the present invention the radionuclide is ^{99m}Tc. In one or more embodiments of the present invention the radionuclide is ¹¹¹In. In one or more embodiments of the present invention the radionuclide is ²⁰³Pb. In one or more embodiments of the present invention the radionuclide is ¹⁵²Tb. In one or more embodiments of the present invention the radionuclide is ¹⁵⁵Tb. In one or more embodiments of the present invention the radionuclide is ⁸³Sr. In one or more embodiments of the present invention the radionuclide is ⁸⁶Y.

These radionuclides can be combined in the particles of the present invention, so the particle comprises one, two or more of the above-mentioned radionuclides. This can happen by natural causes where a radionuclide decays and therefore becomes its natural progeny. Such situation can for example happen when ²²⁴Ra is the parent radionuclide, ²²⁰Rn (the daughter radionuclide), ²¹⁶Po (the granddaughter radionuclide), and ²¹²Pb (the great granddaughter radionuclide). ²²⁰Rn, ²¹⁶Po and ²¹²Pb are therefore all considered progeny radionuclides of ²²⁴Ra and will due to the natural decay of ²²⁴Ra automatically be present in the particles in certain amounts.

Two or more radionuclides can also be comprised in the particle because it can be beneficial to have higher amounts than from the natural decay for the intended use of the particle. This can for example happen if ²²⁴Ra and ²¹²Pb are mixed for the formation of the particles. There will in this situation be a higher level of ²¹²Pb in the particle than there would be if the particle was prepared with purified ²²⁴Ra.

The amount of radionuclide used per patient dosage may be in the range of 1 kBq to 10 GBq more preferably 100 kBq to 100 MBq, event more preferably range is 0.5 MBq to 25 MBq. Range dosage can be 10 MBq to 10 GBq per patient dose. Range dosage can be 10 MBq to 5 GBq per patient dose. The ranges can be for beta emitters, alpha emitters or combinations hereof. The ranges can be for therapy or imaging. Dosage will depend on the cancer type, and for example how aggressive the disease is. In one embodiment is the dosage 10-100 kBq/kg, such as 20-50 kBq/kg. In another embodiment is the dosage 10-1000 kBq/kg, such as 25-300 kBq/kg. In a further embodiment the is the dosage 100-500 kBq/kg, such as 150-300 kBq/kg. In one embodiment the dosage is 1-100 MBq/kg, such as 5-20 MBq/kg. In another embodiment the dosage is 1-1000 MBq/kg, such as 10-50 MBq/kg. In a further embodiment the is the dosage 100-500 MBq/kg, such as 150-300 MBq/kg.

In one embodiment of the present invention is the pharmaceutical composition prepared with an amount of radionuclide that is 1 kBq to 10 GBq per dosing. For instance, if 100 patient doses are produced in one batch per day this could be made up of a total of 1-10 GBq divided into 100 single dosing vials or ready to use syringes.

In another embodiment of the present invention is the pharmaceutical composition prepared with an amount of radionuclide that is suitable for multidose industrial scale production e.g., 50 MBq to 100 GBq.

Thus, the compositions of the present invention can be prepared with an amount of radionuclide that is 1 kBq to 10 GBq per dosing or with an amount of radionuclide that is 50 MBq to 100 GBq suitable for multidose industrial scale production.

### Morphology and Compositions

The particles can have a variety of characteristics, and the size of the particles can vary depending on the intended uses and applications.

The type of crystals may be any known form of degradable compound and sizes varying from 1 nm to 500 μm may be used. The size can be in the range of 100 nm to 50 μm and further preferably is size in the range of 1-10 μm. In one preferred embodiment is the size 1-10 μm. In another preferred embodiment the size is 100 nm to 5 μm, and in another 10-100 nm. In another preferred embodiment, the size is 1-20 μm, and in another 2-10 μm.

An aspect relates to a composition comprising one or more particles according to the present invention. The composition may be a particle suspension comprising monodisperse or polydisperse particles comprising a degradable compound, a radionuclide and a phosphorus additive.

One or more embodiments of the present invention relates to the use of the particles of the present invention, where the radionuclide is either surface labeled by the radionuclide, inclusion labeled as part of particle volume, or a surface labeled particle that after radiolabeling is covered with a layer of material to protect the radiolabeled surfaces and prevent radionuclide release. The particle of the present invention can then become a radionuclide labeled particle whereby a layer of material has been added to cover the original surface to encapsulate the radionuclide. Example 5 has described these various types of preparing the particles.

The surface labelling can be performed as an adsorption of the radionuclide to the crystal particles driven by the affinity of the elements or the labelling can be performed as co-precipitation where additional inorganic compounds aid the precipitation process. A chelator can be use in this process, and the chelator can be incorporated in the particle or on the surface of the particle.

An aspect of the present invention relates to a composition comprising a particle comprising a degradable compound and a radionuclide, wherein a phosphorus containing additive is comprised in the composition. The composition can be a suspension of particles. The phosphorus containing additive can be incorporated into the particle. The phosphorus containing additive can be associated with the surface of the particle or be present in the surroundings of the particle, i.e. in the composition or suspension that the particle is part of. Thus, one aspect of the present invention relates to a composition or suspension comprising a particle, wherein the particle comprises a degradable compound, a radionuclide and a phosphorus containing additive, and wherein the phosphorus containing additive is associated with the particle by being present in the composition or suspension. The phosphorus containing additive can be as part of the particle. The presence can be on the surface of the particle. The presence can be as part of the composition or suspension of particles. The presence can also be as part of the particle and as part of the composition or suspension of particles.

One or more embodiments of the present invention relate to a particle suspension which is a mixture of a solid phase and a liquid phase. The phosphorus containing additive may either be in the liquid phase. The containing phosphorus additive can be in the solid phase. The phosphorus containing additive can be the solid and the liquid phases. In the solid phase the phosphorus containing additive can be on the surface or embedded in the particles or both on the surface or embedded in the solid phase. The solid phase might be made out of nanoparticles, microparticles or a combination those two. The radionuclide may be associated with the surface of the particle or embedded in the volume or bulk of the particle, or both. The solid phase can therefore comprise a particle comprising a degradable compound and a radionuclide, with or without a phosphorus containing additive, but the phosphorus containing additive will always be in the liquid phase if it is not part of the solid phase. The degradable compound, radionuclide and phosphorus containing additive can be any of those disclosed herein.

The phosphorus containing compound may or may not complex radionuclide.

The composition of the present invention is preferably an aqueous composition. Thus, in this embodiment the liquid phase is an aqueous phase. The composition can be a saline composition. The composition can also be an alcohol composition. The composition can be a gel-matrix composition. The composition of the present invention can be a suspension of the particles of the present invention.

Thus, a further aspect of the present invention relates to a composition or a pharmaceutical composition comprising one or more particles according to the invention and a diluent, carrier, surfactant, deflocculant and/or excipient.

Acceptable carriers and pharmaceutical carriers include but are not limited to non-toxic buffers, fillers, isotonic solutions, solvents and co-solvents, anti-microbial preservatives, anti-oxidants, wetting agents, antifoaming agents and thickening agents etc. More specifically, the pharmaceutical carrier can be but are not limited to normal saline (0.9%), half-normal saline, Ringer's lactate, dissolved sucrose, dextrose, e.g. 3.3% Dextrose/0.3% Saline. The physiologically acceptable carrier can contain a radiolytic stabilizer, e.g. ascorbic acid, human serum albumin, which protect the integrity of the radiopharmaceutical during storage and shipment.

The particles may be dispersed in various buffers compatible with medical injections, e.g., dissolved salts and/or proteins and/or lipids and or sugars.

The pharmaceutical compositions can comprise a multitude of particles. These can be the same or different.

### Medical Applications

The particles and compositions or the present invention can be used as radiotherapeutic compounds and/or radiotherapeutic mixtures (compositions and solutions).

An aspect of the invention relates to the particle, composition or pharmaceutical composition of the present invention for use as a medicament. An aspect of the invention relates to the particle, composition or pharmaceutical composition of the present invention for use in the treatment of cancer.

Parenteral injection is a term that encompasses at least intravenous (IV), intramuscular (IM), subcutaneous (SC) and intradermal (ID) administration. Thus, one or more embodiments of the present invention relates to the use of particle, composition or pharmaceutical composition of the present invention in an administration that comprises parenteral injection. One or more embodiments of the present invention relates to the use of particle, composition or pharmaceutical composition of the present invention in an administration that comprises intravenous (IV) administration. One or more embodiments of the present invention relates to the use of particle, composition or pharmaceutical composition of the present invention in an administration that comprises intramuscular (IM), administration. One or more embodiments of the present invention relates to the use of particle, composition or pharmaceutical composition of the present invention in an administration that comprises subcutaneous (SC) administration. One or more embodiments of the present invention relates to the use of particle, composition or pharmaceutical composition of the present invention in an administration that comprises intradermal (ID) administration. One or more embodiments of the present invention relates to the use of particle, composition or pharmaceutical composition of the present invention in an administration that comprises intra-tumor administration.

Medical uses of the particles of the present invention includes human or veterinary use can be in (1) Intracavitary therapy (2) radioembolization (3) radiosynovectomy (4) imaging (5) as a medical device.

Intracavitary therapy may include treatment of e.g., intraperitoneal cancers, intracranial cancers, pleural cancers, bladder cancers, cardiac cancers, cancers in the subarachnoid cavity. Examples of cavities where the particles may be used is cranial cavity, thoracic cavity, lung cavity, spinal cavity, pelvic cavity, pericardium, pleural cavity, bladder cavity or a combination of these including cancers spreading on the peritoneum or meninges and organs within any of these cavities. In one embodiment of the present invention is the cancer selected from the group consisting of intraperitoneal cancers, intracranial cancers, pleural cancers, bladder cancers, cardiac cancers, and cancers in the subarachnoid cavity. In one embodiment of the present invention is the cancer selected from the group consisting of metastatic cancer, lung cancer, ovarian cancer, colorectal cancer, stomach cancer, pancreatic cancer, breast cancer, neoplastic meningitis, peritoneal cancer, pleural effusion, malignant mesothelioma, breast cancer, sarcomas, brain cancers like glioblastoma and astrocytoma, prostate cancer, bladder cancer, and liver cancer. One or more embodiments of the present invention relates to the use according to the invention, wherein the cancer is metastatic cancer. One or more embodiments of the present invention relates to the use according to the invention, wherein the cancer is lung cancer. One or more embodiments of the present invention relates to the use according to the invention, wherein the cancer is ovarian cancer. One or more embodiments of the present invention relates to the use according to the invention, wherein the cancer is colorectal cancer. One or more embodiments of the present invention relates to the use according to the invention, wherein the cancer is stomach cancer. One or more embodiments of the present invention relates to the use according to the invention, wherein the cancer is pancreatic cancer. One or more embodiments of the present invention relates to the use according to the invention, wherein the cancer is breast cancer. One or more embodiments of the present invention relates to the use according to the invention, wherein the cancer is neoplastic meningitis. One or more embodiments of the present invention relates to the use according to the invention, wherein the cancer is peritoneal cancer. One or more embodiments of the present invention relates to the use according to the invention, wherein the cancer is pleural effusion. One or more embodiments of the present invention relates to the use according to the invention, wherein the cancer is pleural effusion. One or more embodiments of the present invention relates to the use according to the invention, wherein the cancer is malignant mesothelioma. One or more embodiments of the present invention relates to the use according to the invention, wherein the cancer is breast cancer. One or more embodiments of the present invention relates to the use according to the invention, wherein the cancer is prostate cancer. One or more embodiments of the present invention relates to the use according to the invention, wherein the cancer is pericardial cancer One or more embodiments of the present invention relates to the use according to the invention, wherein the cancer is sarcoma. One or more embodiments of the present invention relates to the use according to the invention, wherein the cancer is brain cancers like glioblastoma and astrocytoma. One or more embodiments of the present invention relates to the use according to the invention, wherein the cancer is bladder cancer. One or more embodiments of the present invention relates to the use according to the invention, wherein the cancer is liver cancer.

An aspect of the invention relates to the particle, composition or pharmaceutical composition of the present invention for use in the treatment of cancer, wherein the cancer is selected from the group consisting of intraperitoneal cancers, intracranial cancers, pleural cancers, bladder cancers, cardiac cancers, cancers in the subarachnoid cavity, non-cavitary targets such as melanoma, non-small-cell-lung cancer.

In a special embodiment for the use of the particles of the present is treatment or amelioration of a disease which is an infection or inflammation rather than or in combination with cancer. The inflammation can for example be arthritis.

In one embodiment of the present invention is the infection selected from the group consisting of a bacterial infection and viral infection.

Radioembolization may include treatment of primary or metastatic cancer in an organ e.g., the liver by administering the particles of the present invention to a blood vessel leading to a tumor in the liver or another solid organ infiltrated by tumor tissue.

Radiosynovectomy for joint disorders including chronic inflammations is targeted radiation treatment for painful joint diseases using radioactive substances. Its use includes treatment of hemophilic arthritis.

Today it is based on beta-particle emitting compounds used for inflammatory or rheumatoid diseases, or synovial arthrosis of various joints, in particular of the knee, hand and ankle. The particles described herein which are degradable could be very useful in radiosynovectomy.

The particles are preferably administered by local injection, e.g. intracavitary. In a special embodiment the particles are injected directly into a tumor.

Another aspect of the present invention relates to a method of treatment or amelioration comprising administration of the particles or the pharmaceutical composition of the present invention to an individual in need thereof.

The compositions of the present invention can be suitable for parenteral use, for instance intravenous, intracavitary and/or intratumor injections.

In an aspect of the present invention is the particle according to the present invention a medical device or is comprised in a medical device.

A medical device is any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings for the purpose of: Diagnosis, prevention, monitoring, treatment or alleviation of disease; Diagnosis, monitoring, treatment, alleviation of or compensation for an injury or handicap; Investigation, replacement or modification of the anatomy or of a physiological process; Control of conception; and which does not achieve its principal intended action in or on the human body by pharmacological, immunological or metabolic means, but which may be assisted in its function by such means

Medical devices vary according to their intended use and indications. Examples range from simple devices such as tongue depressors, medical thermometers, and disposable gloves to advanced devices such as computers which assist in the conduct of medical testing, implants, and prostheses.

According to the FDA is medical device “an instrument, apparatus, implement, machine, contrivance, implant, in vitro reagent, or other similar or related article, including a component part, or accessory which is: recognized in the official National Formulary, or the United States Pharmacopoeia, or any supplement to them, intended for use in the diagnosis of disease or other conditions, or in the cure, mitigation, treatment, or prevention of disease, in man or other animals, or intended to affect the structure or any function of the body of man or other animals, and which does not achieve any of its primary intended purposes through chemical action within or on the body of man or other animals and which is not dependent upon being metabolized for the achievement of any of its primary intended purposes.”

The present particles are not being metabolized nor do they have significant chemical action within the body. The particles are carriers of radioactivity that are designed not be metabolized or have any chemical action within the body, and this allows for radiotherapy with very limited unwanted side-effects, such as toxicity.

Thus, in one embodiment is the term “medical device” understood as FDAs definition above.

### Imaging

Cancer imaging is an umbrella term that covers the many approaches used to research and diagnose cancer. Originally used to diagnose and stage the disease, cancer imaging is now also used to assist with surgery and radiotherapy, to look for early responses to cancer therapies and to identify patients who are not responding to treatment. One element is nuclear imaging involving the application of radioactive substances in the diagnosis and monitoring of disease. Nuclear medicine imaging, in a sense, is “radiology done inside out” or “endoradiology” because it records radiation emitting from within the body rather than radiation that is generated by external sources like X-rays. In addition, nuclear medicine scans differ from radiology, as the emphasis is not on imaging anatomy, but on the function. For such reason, it is called a physiological imaging modality. Single photon emission computed tomography (SPECT) and positron emission tomography (PET) scans are the two most common imaging modalities in nuclear medicine.

In nuclear medicine, an approach where diagnostic imaging and therapy is combined is termed theranostics. Visualization of the distribution of a radiolabeled particle can help predict if the patients will have a benefit from the treatment and/or to decide on the optimal dose to be administered. Even if some therapeutic radionuclides have decay properties allowing them to be imaged, only a few have decay characteristics allowing for specific and precise determination of radioactivity distribution in the body. Therefore, the idea of radiolabeling an agent with a surrogate for the therapeutic radionuclide was established. For in vivo diagnostic imaging, short-lived gamma- or positron emitters like ^{99m}Tc, ¹⁸F and ⁶⁷Ga are commonly used. Despite optimal imaging properties, using these radionuclides is not feasible in all cases because of different chemistry than the therapeutic radionuclide. In addition, significantly different half-lives between the diagnostic and therapeutic radionuclide can complicate the interpretation of the biodistribution. Therefore, ¹¹¹In has for instance been used as a surrogate for ⁹⁰Y, since they are both trivalent metals with similar half-lives (2.8 vs 2.7 days). The idea of using a pair of radionuclides of the same element, one with decay characteristics suitable for therapy and the other for imaging, a so-called theranostic radionuclide pair, has also emerged. Examples of such are ⁸⁶Y/⁹⁰Y ⁶⁴Cu/⁶⁷Cu, ⁸³Sr/⁸⁹Sr, ¹⁵²Tb/¹⁶¹Tb and ²⁰³Pb/²¹²Pb.

Thus, an aspect of the invention relates to the particle, composition or pharmaceutical composition of the present invention for use in imaging. In one or embodiments of the present invention the imaging is nuclear medicine imaging.

### Combinational Therapy

The degradable particle can comprise many different additional compounds. These can serve various purposes included targeting, stability, solubility and rate of degradation.

In one embodiment of the present invention, the particle comprises one or more compounds selected from the group consisting of a monoclonal antibody, a polyclonal antibody, a radioimmunoconjugate, an immunoconjugate, a chelate antibody conjugate, vitamins including folate and folate derivatives, peptides, minibodies, and affibodies.

In an embodiment, the particles comprise antibody, antibody fragment or protein or peptide or vitamin derivative (targeting conjugate) with affinity for receptors including antigens on the tumor cells.

In another embodiment, the particles comprise radiolabeled antibody, antibody fragment or protein or peptide or vitamin derivative (targeting conjugate) with affinity for receptors including antigens on the tumor cells whereby the labeled particles will give a general particle radiation field on the surfaces, and the labeled antibody or similar gives a specific alpha particle dose to the tumor cells by receptor or antigen binding.

The radionuclides in the present invention can be conjugated to a targeting molecule by using bifunctional chelators.

These could be cyclic, linear or branched chelators. Particular reference may be made to the polyaminopolyacid chelators which comprise a linear, cyclic or branched polyazaalkane backbone with acidic (e.g. carboxyalkyi) groups attached at backbone nitrogens.

Examples of suitable chelators include DOTA derivatives such as p-isothiocyanatobenzyl-1,4,7,10-tetraazacyclododecane-1,4,7, 10-tetraacetic acid (p-SCN-Bz-DOTA) and the tetra primary amide variant of this DOTA compound, termed TCMC, and DTPA derivatives such as p-isothiocyanatobenzyl-diethylenetriaminepenta-acetic acid (p-SCN-Bz-DTPA), the first being cyclic chelators, the latter linear chelators.

Metallation of the complexing moiety may be performed before or after conjugation of the complexing moiety to the targeting moiety.

The radiolabeling procedure will in general be more convenient in terms of time used etc. if the chelator is conjugated to the antibody before the radiolabeling takes place.

An aspect of the invention relates to the particle, composition or pharmaceutical composition of the present invention for use according to the present invention, which is used in combination with other cancer therapies.

Examples of therapies are chemotherapy like taxanes (e.g. paclitaxel, docetaxel), platins (e.g. carboplatin, cisplatin), doxorubicin, mitomycin. Other examples are DNA repair inhibitors, such as PARP inhibitors (e.g. Olaparib, Rucaparib, Niraparib, Talazoparib, Veliparib, Pamiparib, CEP 9722 E7016, and 3-Aminobenzamide). Further examples are radioimmunotherapies.

The DNA repair inhibitor can be selected from the group consisting of a poly (ADP-ribose) polymerase inhibitor (PARPi), a MGMT inhibitor, a DNA-dependent protein kinase inhibitor (DNA-PK inhibitor), a ataxia telangiectasia and Rad3-related (ATR) kinase inhibitor, a ataxia telangiectasia mutated (ATM) kinase inhibitor, a Wee1 kinase inhibitor, and a checkpoint kinase 1 and 2 (CHK1/2) inhibitor.

In one or more embodiments of the present invention, the PARPi is selected from the group consisting of Olaparib, Rucaparib, Niraparib, Talazoparib, Veliparib, Pamiparib, CEP 9722, E7016, and 3-Aminobenzamide.

The PARPi can be Olaparib. The PARPi can be Rucaparib. The PARPi can be Niraparib. The PARPi can be Talazoparib. The PARPi can be Veliparib. The PARPi can be Pamiparib. The PARPi can be CEP 9722. The PARPi can be E7016. The PARPi can be 3-Aminobenzamide.

### Method for Preparing the Particles

An aspect of the invention relates to a method for preparing a particle according to the present invention, the method comprising bringing a degradable compound, a radionuclide, and a phosphorus containing additive in contact with each other with or without using a carrier for the radionuclide. A degradable compound and a radionuclide can form a first particle in an initial step, which can be followed by an additional step of adding a degradable compound to the already radiolabeled particle, thereby giving a layered particle. Subsequently a phosphorus containing additive is added to stabilize the particle. The phosphorus containing additive can in this process become part of the particle and remain in the composition where the particle is. The phosphorus containing additive can also both be part of the particle and remain in the composition comprising the particle. Examples of different particle preparations can be seen in examples 1 and 5. Another example of a particle preparation can be seen in Example 18.

Phosphorus containing compounds such as phosphonates and phosphates can be applied as additives to stabilize crystal particles, i.e. they are the phosphorus containing additives described herein. They can be added to particles, such as crystal particles, during the formation of these, after the particles have been formed, after labeling or to the final formulation in order to achieve the size control. The product can be sterilized by autoclaving and the additives can be included before or after this process. The size controlling additive can also be used as a component of a kit used to prepare a final product.

Example 18 describes calcium carbonate microparticles produced with addition of glycerol in the spontaneous precipitation reaction in order to produce smaller microparticles (CaCO₃ SMPs).

Thus, in embodiments an additional size controlling additive is added in the preparation of the particle. In embodiments the additional size controlling additive is an alcohol such as but not limited to a diol or triol. In embodiments the additional size controlling additive is selected from the group consisting of Etylene glycol (EG), PEG, Glycerol and Dextran, Ethanol. In embodiments the additional size controlling additive is Glycerol.

The phosphorus containing compounds can stabilize crystal particles of monodisperse and polydisperse particles. The particles will typically be polydisperse because they are made in solution, but they can share characteristics, such as having a size within a given similar range.

Thus, an aspect of the present invention relates to the use of a phosphorus containing additive for size control of particles comprising a degradable compound. Another aspect of the present invention relates to the use of a phosphorus containing additive for stabilizing particles comprising a degradable compound. These particles can be the particles of the present invention, before or after a radionuclide is added. The particles can therefore be the degradable compound itself. In one embodiment of the present invention, the degradable compound is a crystal particle.

A solution or composition comprising radionuclide, e.g. a ²²⁴Ra solution or composition with progeny ²¹²Pb in mixture could be pretreated with chelate-antibody conjugate to complex ²¹²Pb prior to particle labeling to produce a two-component therapeutic system containing a radioimmunoconjugate for ²¹²Pb antigen-specific treatment and alpha emitter, e.g. ²²⁴Ra-labeled particles for a general cavity treatment. A biologic compound, such as an antibody, can also be part of these particles. These can then subsequently be mixed with a phosphorus containing additive to give a size controlled particle. The particle of the composition could also be ²¹²Pb-labeled particles in a composition with a ²¹²Pb antigen-specific treatment.

An embodiment relates to a three-component system or kit comprising a radionuclide such as a radioimmunoconjugate for antigen-specific treatment, a degradable compound, and a phosphorus containing additive.

The preferable way to use this would be by a kit containing a vial A with chelate-conjugated antibody and a vial B with radionuclide, e.g. ²²⁴Ra in equilibrium with daughter nuclides, and a vial C with microparticles, whereby the content of A is added to vial B, or vice versa, and incubated from a few minutes to a few hours before the mixture is transferred to vial C for further incubation for a few minutes to a few hours before being mixed with the phosphorus containing additive and subsequently transferred to a syringe and injected into the patient.

This principle could significantly reduce the level of for example ²¹²Pb-radioimmunoconjugate needed for therapy since ²²⁴Ra-particles is expected to contribute strongly to the antitumor activity in such a system.

Another aspect of the present invention relates to a kit comprising a nano- or micro-particle according to the present invention, and optionally instructions to use the kit.

In one embodiment of the present invention, the kit comprises a chelator-conjugated molecule, including monoclonal antibody.

The current methods and product allow for centralized production and shipment to the end user since the radionuclide has several days half-life. Another aspect of the presented invention is the use of a biodegradable particle that slowly dissolves into calcium and carbonate thereby producing small amounts of products that are already abundantly present in the body. It is also noteworthy of the following feature: When a radionuclide, e.g. ²²⁴Ra is absorbed the degradable compound, such as calcium carbonate, there is a significant release of short living ²²⁰Rn (t_{1/2}=56 s) which will together with the ultra-short lived ²¹⁶Po (t_{1/2}=0.16 s) produce two alpha particles before decaying to the longer lived beta emitter ²¹²Pb (t_{1/2}=10.6 h). Lead has a very high precipitability with for example calcium carbonate so the ²¹²Pb in the i.p. fluid will tend to re-associate to the particles diminishing leakage of ²¹²Pb into the systemic circulation.

It is therefore a very special technical feature that ²²⁴Ra decays into a gas that can diffuse out of the particle and afterwards decay further into ²¹²Pb that precipitates with calcium carbonate.

Pre-produced particles and subsequent surface sedimentation or radionuclide co-sedimentation for deeper inclusion of radionuclide are two methods useful for producing a therapeutic product. Once the radionuclide and the degradable compound has formed a first particle, the phosphorus containing additive can subsequent size control the first particle and become the particle of the present invention.

The particles may be produced in sizes from nanometers to several tens of micrometers and radiolabeled with high labeling yields and can be stored for several days which is important since it allows centralized production and shipment to the hospitals of ready to use particle suspensions. This can be done without risking the particle integrity and therefore ensure that the particles remain intact and have the size that is needed for the intended use.

As aspect of the present invention relates to a particle produced by any of the methods described herein.

### General

It should be understood that any feature and/or aspect discussed above in connections with the compounds according to the invention apply by analogy to the methods described herein.

The following figures and examples are provided below to illustrate the present invention. They are intended to be illustrative and are not to be construed as limiting in any way.

### BRIEF DESCRIPTION OF THE FIGURES

FIG. 1: Ex vivo tissue distribution of ²²⁴Ra 1 day after intraperitoneal administration of different variants of ²²⁴Ra—CaCO₃-microparticles: 1 and 5 mg surface labeled without additive (A), 1.2, 6 and 12 mg surface labeled and layer protected ²²⁴Ra—CaCO₃-microparticles in EDTMP (B), surface labeled ²²⁴Ra—CaCO₃-microparticles washed with different amounts of SHMP (C) and inclusion labeled ²²⁴Ra—CaCO₃-microparticles in EDTMP (D). The data are shown as a bar representing the median percent injected dose per gram tissue of collected organs and tissues in addition to symbols representing the individual data points for each mouse.

FIG. 2: Ex vivo tissue distribution of radioactivity 1 day after intraperitoneal administration of cationic ²²⁴Ra. The data are shown as a bar representing the median percent injected dose per gram tissue of collected organs and tissues in addition to symbols representing the individual data points for each mouse.

FIG. 3: Kaplan-Meier survival plots of athymic nude Foxnu mice inoculated intraperitoneally with 1×10⁶ ES-2 cells and treated one day later with intraperitoneal injections of 0.9% NaCl or five different variants of ²²⁴Ra-labeled CaCO₃ microparticles. N=5-6 animals per group.

FIG. 4: Kaplan-Meier survival plots of BALB/c mice inoculated intraperitoneally with 5×10⁴ CT26.WT cells and treated one day later with intraperitoneal injections of 0.9% NaCl or four different variants of ²²⁴Ra-labeled CaCO₃ microparticles. N=7-8 animals per group.

FIG. 5. Size distribution of CaCO₃ microparticles. (a) Unlabeled, non-radioactive CaCO₃-MPs (MPs: microparticles) suspended in saline and autoclaved on day zero with varying EDTMP % (w/w) compared with the unautoclaved CaCO₃-MPs used as raw material. (b) Surface ²²⁴Ra labeled CaCO₃-MPs with varying EDTMP % (w/w) compared with the unautoclaved CaCO₃-MPs used as raw material. (c) Autoclaved and layer encapsulated ²²⁴Ra surface labeled CaCO₃-MPs with varying EDTMP % (w/w) compared with the unautoclaved CaCO₃-MPs used as raw material. (d) Comparison of surface labeled and layer encapsulated surface labeled MPs, excerpts from (b) and (c) Dv: volumetric diameter, with Dv10 being the 10-percentile, etc.

FIG. 6. Sedimentation rate of non-radioactive autoclaved CaCO₃ microparticles of different sizes. (a) Comparison of mock surface labeled MPs suspended in 0.9% NaCl with and without EDTMP after the suspension was allowed to sit for 30 seconds and 4 minutes, respectively. (b) Assessment of the turbidity of suspended CaCO₃-MPs of different sizes, all mock surface labeled; layer encapsulated MPs are indicated by the line marked with “x”. Dv: volumetric diameter, with Dv50 being the 50-percentile.

FIG. 7. Radiochemical analysis of adsorbed ²¹²Pb and ²²⁴Ra on CaCO₃ microparticles (MPs) with varying EDTMP concentrations. Symbols represent independent samples and EDTMP concentration is relative to CaCO₃. (a) Percentage adsorbed ²¹²Pb (% RCP) on the MPs on different days after labeling, surface labeled MPs vs layer encapsulated surface labeled MPs. (b) Percentage adsorbed ²²⁴Ra on the MPs (% RCP) on different days after labeling, surface labeled MPs vs layer encapsulated surface labeled MPs. (c) Percentage ²¹²Pb-EDTMP in the liquid phase after subtracting unspecific migration by ²¹²Pb2+ in 0.9% NaCl in the ITLC setup on different days after labeling, surface labeled MPs vs layer encapsulated surface labeled MPs.

FIG. 8. Biodistribution of layer encapsulated ²²⁴Ra surface labeled CaCO₃ microparticles one day after i.p. injection. Bars represent the median and symbols represent individual animals. Animals that were treated with equal or near-equal mass doses have been pooled into one group. (a) Percentage injected dose per gram tissue of ²²⁴Ra. (b) Percentage injected dose per gram tissue of ²¹²Pb from the same samples as in (a). There is missing data for ²²⁴Ra and ²¹²Pb for one skull in the ²²⁴RaCl₂ group and for ²¹²Pb for one blood sample in the 12 mg group.

FIG. 9. Distribution of ²⁰⁸Pb and ²¹²Pb on layer encapsulated microparticles with 2.5% (w/w) EDTMP as a function of days after surface labeling with ²²⁴Ra. It is assumed that ²²⁴Ra is in equilibrium with its daughters, with a total amount of 200 atoms (or a.u.) for each of ²²⁴Ra, ²¹²Pb, and ²⁰⁸Pb on day zero. An equal proportion of ²⁰⁸Pb and ²¹²Pb adsorbed on MPs (RCP) versus in the solution, is assumed, with 94% RCP from day 0-3, 81% RCP from day 4-6, and 79% RCP on day 7. One ²⁰⁸Pb atom is produced per ²²⁴Ra decay, while the total number of ²¹²Pb atoms is found by using the Bateman equation, taking both ²²⁴Ra and ²¹²Pb decay into account.

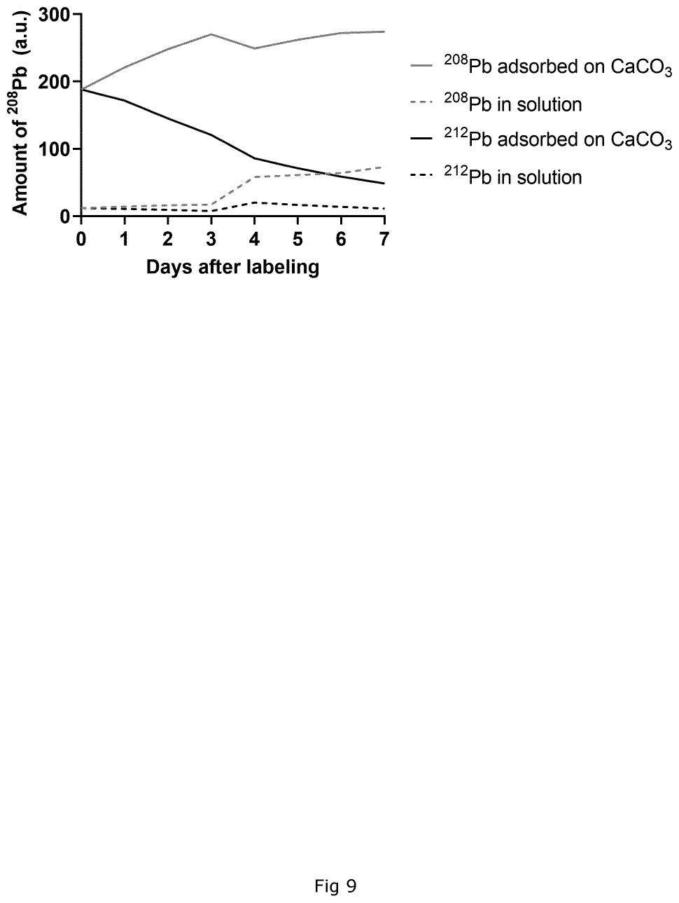

FIG. 10. Percentage adsorption of ²¹²Pb²⁺ and ²¹²Pb-EDTMP to non-radioactive mock labeled CaCO₃-MPs, surface labeled MPs and layer encapsulated surface labeled MPs. EDTMP concentrations indicate the relative concentration in the MP suspension with respect to grams per gram of CaCO₃.

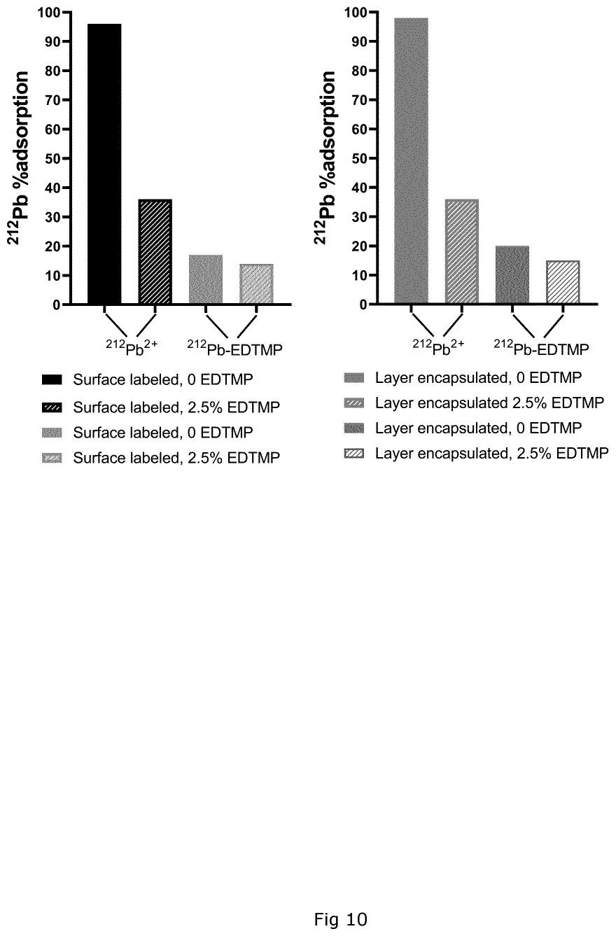

FIG. 11. Radiochemical properties of various CaCO₃ particles in suspension after autoclaving and labeling with ²¹²Pb. Median diameters are based on laser diffraction measurements. (a) Percentage radiolabeling yield (same quantity as RCP) as function of time after ²¹²Pb was added. (b, c) Percentage RCP as function of particle size and/or type of phosphonate on the same day as labeling (b) or after at least 21 h (c). (d, e) Retained ²¹²Pb on the particle types in (b, c) after incubation in isotonic solution with human serum albumin.

FIG. 12. Biodistribution of ²¹²Pb evaluated as the percentage injected dose per gram of tissue on a designated time following i.p. administration. The bars represent the median and the symbols represent individual animals. (a, b, c)²¹²Pb—CaCO₃ microparticles in pamidronate compared with free ²¹²Pb²⁺ administered as ²¹²PbCl₂, with the percentage injected dose per gram of tissue after (a) 2 h, (b) 6 h, and (c) 24 h. (d) Comparison of the biodistribution of ²¹²Pb—CaCO₃ microparticles in pamidronate to smaller microparticles (SMPs) in pamidronate produced in glycerol presence. Data on urine and/or bladder is not reported in (a, c, d) due to missing data for 2/3 mice in one of the groups, while one urine sample is missing in (b, d).

FIG. 13. Survival plots of athymic nude Foxnu mice inoculated intraperitoneally with ES-2 cells and treated one day later with intraperitoneal injections of 0.9% NaCl or different activity doses of ²¹²Pb—CaCO₃ MPs in pamidronate.

### EXAMPLES

### Example 1—Preparation of Microparticles

Crystalline CaCO₃ microparticles subsequently used for surface labeling with ²²⁴Ra were prepared by a spontaneous precipitation method based on the protocol described by Volodkin et al. 2004. Equal volumes of 0.33 M Na₂CO₃ (Merck, Darmstadt, Germany) and 0.33 M CaCl₂) (Merck) was vigorously mixed with an overhead stirrer (Eurostar 20, IKA®-Werke GmbH & Co. KG, 120 Staufen, Germany) before the precipitated microparticles were collected by centrifugation. The precipitate was washed in ph.Eur water and dried in a heated vacuum oven. The microparticles had mainly spherical geometry, and the median diameter was 4 to 7 μm when measured by a laser diffraction particle size analyzer (Mastersizer 3000, Malvern Instruments Ltd, Worcestershire, UK).

Microparticles were also prepared by mixing of stock solutions of up to 1 M of Na₂CO₃ and CaCl₂) with ²²⁴Ra present to produce inclusion labeled microparticles as described further in Example 5. Example 5 also describe how in some applications; a layer of CaCO₃ was precipitated on the surface of already labeled microparticles to make a thin encapsulation for surface protection.

### Example 2—Use of Phosphorus Containing Additive for Size Control

Phosphorus containing compounds such as phosphonates and phosphates can be applied as additives to stabilize crystal particles. They can be added to crystal particles during the formation of these, after the crystal particles have been formed, after labeling or to the final formulation in order to achieve the size control. The product can be sterilized by autoclaving and the additives can be included before or after this process. The size controlling additive can also be used as a component of a kit used to prepare a final product.

Phosphonates and phosphonic acids are organophosphorus compounds containing C—PO(OH)₂ or C—PO(OR)₂ groups (where R=alkyl, aryl). Phosphonic acids, typically handled as salts, are generally non-volatile solids that are poorly soluble in organic solvents, but soluble in water and common alcohols. Bisphosphonates include Etidronate (HEDP), Clodronate, Tiludronate, Pamidronate, Neridronate, Olpadronate, Alendronate, Ibandronate, Risedronate, Zoledronate. Polyphosphonates include EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid)

A phosphoric acid, in the general sense, is a phosphorus oxoacid in which each phosphorus atom is in the oxidation state +5, and is bonded to four oxygen atoms, one of them through a double bond, arranged as the corners of a tetrahedron. Removal of the hydrogen atoms as protons H+ turns a phosphoric acid into a phosphate anion. Partial removal yields various hydrogen phosphate anions. Phosphates include orthophosphate, linear oligophosphates and polyphosphates, such as pyrophosphate, tripolyphosphate and triphosphono phosphate, and cyclic polyphosphates such as sodium hexametaphosphate (SHMP).

### Example 3—Production of ²²⁴Ra and ²¹²Pb

The ²²⁴Ra-generator was prepared by mixing a ²²⁸Th source with an actinide resin and loading it on a column. A source of ²²⁸Th in 1 M HNO₃ was purchased from Eckert & Ziegler (Braunschweig, Germany) or Oak Ridge National Laboratory (TN, USA), and an actinide resin based on the DIPEX® Extractant was acquired from Eichrom Technologies LLC (Lisle, IL) in the form of a pre-packed cartridge of 2 mL. The material in an actinide resin cartridge was extracted and the resin was preconditioned with 1 M HCl (Sigma-Aldrich). A slurry of approximately 0.25 mL actinide resin, 0.25 mL 1 M HCl and 0.1 mL ²²⁸Th in 1 M HNO₃ was prepared in a vial (4 mL vial, E-C sample, Wheaton, Millville, NJ) and incubated with gentle agitation for immobilization of ²²⁸Th for 4 h at room temperature and let to rest for a few days. The generator column was prepared in a 1 mL filtration column (Isolute SPE, Biotage AB, Uppsala, Sweden) by first applying 0.2 mL of inactive actinide resin, before the portion containing ²²⁸Th was loaded on top. The inactive resin was introduced in the bottom of the column to serve as a catcher layer if ²²⁸Th was released during operation of the generator. Later, the capacity of the generator was increased. A slurry consisting of 0.4 mL actinide resin, 0.5 mL ²²⁸Th in 1 M HNO₃ and 0.5 mL 1 M HCl was prepared as described above, before it was loaded onto the generator column.

Radium-224 could be eluted regularly from the generator column in 1-2 mL of 1 M HCl. For further purification, the crude eluate from the generator column was loaded directly onto a second actinide resin column. The second column was washed with 1 M HCl. This eluate was evaporated to dryness in a closed system. The vial was placed in a heater block and flushed with N2-gas through a Teflon tube inlet and outlet in the rubber/Teflon septum on the vial. The acid vapor was lead into a beaker of saturated NaOH by a stream of N2-gas. The radioactive residue remaining after evaporation was dissolved in 0.2 mL or more of 0.1 M HCl. A radioisotope calibrator (CRC-25R, Capintec Inc., Ramsey, NJ) was used to measure the total extracted activity in the process.

Lead-212 was produced from ²²⁴Ra via ²²⁰Rn emanation using a novel simplified single chamber diffusion system modified from the method of Hassfjell S., 2001. About 2-20 pL of a ²²⁴RaCl₂ solution (˜250 kBq), prepared as described above, was distributed on the surface of a small paper strip (15×5 mm) attached to a syringe tip that had previously been inserted through the silicone septum of a 3 mL micro reaction vessel glass v-vial (Supelco, Darmstadt, Germany) screw cap. The screw cap was carefully attached to the v-vial, avoiding contact between the paper strip and the v-vial interior surfaces. The sealed v-vial was left to rest overnight in a fume hood. Next, ²²⁰Rn released through air inside the vial from the ²²⁴Ra source, diffused and decayed into ²¹²Pb via its short-lived daughter ²¹⁶Po and was deposited inside the container. After 20 to 28 h, the cap with the paper strip was carefully removed, avoiding ²²⁴Ra contamination of the vial. The ²¹²Pb was subsequently washed off from the glass walls with 1 M HCl and transferred to a new container.

### Example 4—Methods of Measuring Radioactivity

Radioactive samples up to approximately 30 kBq were measured by an automated NaI gamma counter (Hidex, Turku, Finland) in the energy range from 60-110 keV for quantification of ²¹²Pb and 65-345 keV for ²²⁴Ra. As seen in Table 1 the most abundant x- and y-radiation in these energy ranges is from ²¹²Pb. The counts in these windows are assumed to mainly originate from ²¹²Pb with minimal contribution from other nuclides in the series. Radium-224 activity was determined indirectly based on the counts in the 65-345 keV window. This was carried out by re-measuring the samples between 2-4 days after the first measurement, when the initial ²¹²Pb present in the sample had decayed and transient equilibrium had been established. A pure source of ²²⁴Ra reaches equilibrium conditions after approximately 2 days based on the half-lives of ²²⁴Ra and ²¹²Pb.

TABLE 1
Overview of x- and/or γ-lines in the ²²⁴Ra-series with 1% or
higher abundance. The X- and γ-lines are divided into two
columns, one for energies between 65-345
keV and the second for energies above 345 keV.

| | 60-110 keV | | >110 keV | |
|---|---|---|---|---|
| Radionuclide | (Abundance, %) | | (Abundance, %) | |
| ²²⁴Ra | none | | 241.0 | (4.1) |
| ²²⁰Rn | none | | none | |
| ²¹⁶Po | none | | none | |
| ²¹²Pb | 74.8 | (10.3) | 238.6 | (43.6) |
| | 77.1 | (17.3) | 300.1 | (3.3) |
| | 86.8 | (2.1) | | |
| | 87.4 | (4.0) | | |
| | 89.8 | (1.5) | | |
| ²¹²Bi | none | | 727.3 | (6.7) |
| | | | 785.4 | (1.1) |
| | | | 1620.5 | (1.5) |
| ²¹²Po | none | | none | |
| ²⁰⁸Tl | 72.8 | (2.0) | 277.4 | (6.6) |
| | 75.0 | (3.4) | 510.8 | (22.6) |
| | | | 583.2 | (85.0) |
| | | | 763.1 | (1.8) |
| | | | 860.6 | (12.5) |
| | | | 2614.5 | (99.7) |

A radioisotope calibrator (CRC-25R or CRC-55tR Capintec Inc., Ramsey, NJ, USA) was used to measure samples above 50 kBq.

To determine real time distribution of ²²⁴Ra and ²¹²Pb in samples, high purity germanium detectors (HPGe) were used (a Broad Energy Germanium detector BE3830P or a Standard Electrode Coaxial Germanium detector GC3518, Mirion-Canberra, USA). The spectroscopy analysis software package used with the instruments was based on Genie algorithms.

### Example 5—Labeling with ²²⁴Ra: Surface Labeled, Inclusion Labeled and Layer Protected CaCO₃ Microparticles

Radium-224 labeled CaCO₃ microparticles were prepared by three different procedures:
1. Surface labeling by adsorption of ²²⁴Ra onto the surfaces of pre-prepared CaCO₃ microparticles.
2. Inclusion labeling by incorporation of ²²⁴Ra into the CaCO₃ microparticles during their formation.
3. Surface labeled CaCO₃ microparticles where after radiolabeling a layer of CaCO₃ is precipitated onto the original surfaces to encapsulate the radionuclide.

For surface labeling, the CaCO₃ microparticles were washed three times with water and two times with 0.1 M Na₂SO₄ (Merck) immediately before radiolabeling. Radium-224 solution (in 0.1 M HCl with 0.035-0.5 M NH₄OAc) was added to the microparticles under the presence of 0.004 w/w % Ba²⁺ and 0.6 w/w % SO₄²⁻ (Merck) relative to CaCO₃ for coprecipitation of ²²⁴Ra²⁺ on the surface of the microparticles. The radiolabeling process took place in a solution of 0.9% saline (Merck) under orbital rotation for 1.5 h (HulaMixer, Invitrogen, Thermo Fisher Scientific, MA, US).

Inclusion-labeled CaCO₃ microparticles were prepared by rapidly pouring a desired volume of 0.33 M or 1 M CaCl₂ solution containing 0.004 to 0.3 w/w % Ba²⁺ (relative to CaCO₃) and desired amount of ²²⁴Ra into an equal volume of 0.33 M or 1 M Na₂CO₃ solution containing 0.6 to 0.7 w/w % SO₄²⁻ (relative to CaCO₃) while mixing with a magnetic stirrer (BioSan MS3000, Riga, Latvia) or by hand shaking and vortexing. In some applications 1 mg/ml gelatin (Sigma-Aldrich) was added in the CaCO₃ crystallization phase to slow down the crystallization and stored in refrigerator overnight.

Layer protected CaCO₃ microparticles were surface labeled with ²²⁴Ra and excess labeling solution was removed. Subsequently, the microparticles were dispersed in a solution containing CO₃²⁻ and Ca²⁺ ions at concentrations from 0.33-0.66 M under vigorous stirring, in order to precipitate a thin layer of CaCO₃ to cover the original surfaces.

In all cases, excess layering or labeling solution was removed before the CaCO₃ microparticles were washed once or twice with 0.9% NaCl respectively before dispersion in NaCl or NaCl supplemented with a phosphorus compound. In some cases when two washing steps were performed, the phosphorus compound was added after the first washing step and thereby the microparticles were subjected to an additional wash. Finally, the radiolabeled microparticle suspension was autoclaved.

### Example 6—Size Control of CaCO₃ Microparticles with EDTMP, Pamidronate and SHMP

Calcium carbonate microparticles were washed three times with water for injection (WFI), and in some cases additionally two times in 0.1 M sodium sulfate, before suspension in a solution of additive (EDTMP, pamidronate or SHMP) with varying concentration, in 0.9% NaCl. For comparison, samples with zero additive added were suspended in 0.9% NaCl only. Microparticle concentration varied from 30 to 250 mg/ml. The suspension was autoclaved at 121° C. for 21 min. In some cases, measurements were made on CaCO₃ microparticles that were surface labeled with 224Ra as described in Example 5.

Particle size of the autoclaved suspension was measured by use of a laser diffraction particle size analyzer (Mastersizer 3000, Malvern Instruments Ltd, Worcestershire, UK), up to 8 days after suspension had been prepared and stored at room temperature. Additive and additive concentration, microparticle size distribution and day of measurement are summarized Table 2Error! Reference source not found. For comparison, the median volume-based diameter of unprocessed CaCO₃ microparticle raw material when suspended in water is also given. In the absence of additives, the sample preparation immediately caused a 3-fold increase in median particle size, with even larger microparticles after 5 days because of continuing growth in the aqueous medium. By adding the additives EDTMP, SHMP or pamidronate, microparticle size was stabilized with respect to the unprocessed raw material. Impact of additive concentration is most evident for EDTMP; while a concentration of 0.001 g/g relative to CaCO₃ was unable to stabilize the microparticle size 5 days after sample preparation, the size remained stable for EDTMP concentration of >0.013 g/g up to at least 8 days.

TABLE 2
Impact of different phosphorus additives on the size distribution of
CaCO₃ microparticles after autoclaving.

| | | | Median diameter (μm), | | |
|---|---|---|---|---|---|
| | Additive | Median | number of days after sample | | |
| | concentration per | diameter (μm) of | preparation | | |
| Additive | CaCO₃ mass (g/g) | raw material | 0 | 5/6 | 7/8 |
| No additive | n/a | 5.0 | 16.5 | 22.5 | |
| EDTMP | 0.001 | 5.0 | 6.5 | 18.3 | |
| | 0.013 | 5.0 | 3.9 | 3.9 | |
| | 0.014^{(a)} | 4.0 | 3.6 | | 3.4 |
| | 0.024^{(a)} | 4.8 | 4.6 | | 4.7 |
| | 0.046 | 5.0 | 4.2 | 3.9 | |
| SHMP | 0.015^{(a)} | 4.8 | 4.3 | | |
| | 0.030^{(a, *)} | 4.8 | 4.2 | | 4.7 |
| | 0.050^{(b)} | 5.6 | 6.1 | 6.2 | |
| | 0.100^{(b)} | 5.6 | 6.6 | 6.5 | |
| | 0.200 | 5.0 | 4.2 | 4.1 | |
| Pamidronate | 0.008 | 6.7 | 5.2 | | 5.1^{(c)} |
| | 0.009^{(c)} | 5.0 | 4.4 | 4.3 | |
| | 0.016 | 6.7 | 5.2 | | 5.0^{(c)} |

^{(a)}Surface labeled microparticles, * Mock labeling process without ²²⁴Ra.
^{(b)}Includes an additional washing step with WFI after addition of the additive to remove any unbound additive, may influence final additive concentration.
^{(c)}Measured on day 13.

### Example 7—Retention of Radioactivity on CaCO₃ Microparticles In Vitro: Impact of Phosphorus Compounds as Additives

Radiolabeling of CaCO₃ microparticles, including surface labeling, inclusion labeling or layer protected, was performed as described in Example 5. Microparticles were suspended at a concentration from 12.5-250 mg/ml, in 0.9% NaCl with relative additive (EDTMP, pamidronate or SHMP) concentration ranging from 0-0.100 g/g with respect to CaCO₃. The suspension was autoclaved and left in room temperature. To determine the retention of ²²⁴Ra and ²¹²Pb on the microparticles, a sample aliquot was withdrawn at different time points after autoclaving. Subsequently, microparticles were separated from the liquid phase by centrifugation, and the radioactivity in the obtained pellet fraction and supernatant fraction was measured separately using either a HPGe detector or the Hidex automated gamma counter (see Example 4 for details). The percentage retained radioactivity on microparticles was defined as the ratio of activity in the pellet fraction to that in the whole sample before separation.

An overview of the retained radioactivity from both ²¹²Pb and ²²⁴Ra on surface labeled and layer protected CaCO₃ microparticles is given in Table 3 and Table 4 respectively. Addition of a phosphorus compound did not influence the percentage of retained radioactivity on the microparticles the first week after sample preparation, perhaps with the exception of a tendency towards lower degree of ²¹²Pb retention with higher EDTMP amounts. All tested additive amounts were able to control the size of the microparticles (see Example 6).

TABLE 3
Impact of different phosphorus additives on the retention of
radioactivity on surface labeled CaCO₃ microparticles in vitro.

| | Relative additive | % Retained ²¹²Pb/²²⁴Ra on the microparticles, | | | |
|---|---|---|---|---|---|
| | concentration | number of days after sample preparation | | | |
| Additive | (g/g) | 0/1 | 3 | 5 | 7/8 |
| No additive | NA | 99%/86% | 99%/91% | | |
| EDTMP | 0.012 | 80%/97% | | | 77%/95% |
| SHMP | 0.015 | 99%/98% | | | |
| | 0.020 | 100%/100% | 100%/100% | | |
| | 0.100^{(a)} | 97%/86% | 98%/85% | | |
| Pamidronate | 0.009^{(a)} | 100%/90% | | 100%/100% | |

^{(a)}Includes additional centrifugation after addition of the additive to remove any excess, may influence final additive concentration.

TABLE 4
Impact of EDTMP on the retention of radioactivity on layer
protected CaCO₃ microparticles in vitro.

| | | % Retained ²¹²Pb/²²⁴Ra on | | |
|---|---|---|---|---|
| | Relative additive | the microparticles, number | | |
| | concentration | of days after sample preparation | | |
| Additive | (g/g) | 0/1 | 3 | 7 |
| EDTMP | 0.012 | 96%/99% | 96%/99% | |
| | 0.020 | 95%/90% | | 98%/96% |
| | 0.050 | 83%/97% | | 64%/97% |

### Example 8—Biodistribution of Intraperitoneally Administered ²²⁴Ra-Labeled CaCO₃ Microparticles in Mice

Institutionally bred, healthy female athymic nude Foxn^{nu} mice were used. The mice were administered different variants of ²²⁴Ra—CaCO₃-microparticles or cationic ²²⁴Ra intraperitoneally (IP) (see Table 5 for details) and approximately one day post injection blood was collected by cardiac puncture while the animals were under anesthesia. Immediately after, the animals were euthanized before selected organs and tissues were collected, weighed and the radioactivity measured with the Hidex gamma counter. The percent of injected dose per gram tissue of ²²⁴Ra and daughter nuclide with the longest half-life ²¹²Pb (10.6 hours) was estimated. Measurements of the samples as soon as possible after time of sacrifice were used to estimate the amount of ²¹²Pb, whereas a re-measurement minimum 2 days after time of sacrifice were used to determine the amount of ²²⁴Ra. The measured radioactivity was compared directly to the radioactivity in standard samples of the injectate which were measured alongside the samples.

TABLE 5
Overview of performed biodistribution experiments with cationic ²²⁴Ra and different
variants of ²²⁴Ra-labeled CaCO₃ microparticles.

| | Number | Mice | Administered | CaCO₃ | Additive |
|---|---|---|---|---|---|
| | of mice | age | activity | microparticles | concentration per |
| Group | per group | (weeks) | [kBq/mouse] | [mg/mouse] | CaCO₃ mass (g/g) |
| Cationic ²²⁴Ra | 4 | 7- | 3-24 | 0 | n/a |
| Surface labeled | 2 | 5 | 16 | 1 | n/a |
| ²²⁴Ra-CaCO₃- | 3 | 5-48 | 16 | 5 | n/a |
| MP | | | | | |
| Layer protected | 3 | 7 | 6.3 | 1 | 0.012 |
| surface labeled | 3 | 7 | 7.2 | 6 | 0.012 |
| ²²⁴Ra-CaCO₃- | 6 | 7-13 | 13-17 | 12 | 0.012 |
| MP in in | | | | | |
| EDTMP | | | | | |
| Surface labeled | 3 | 11 | 11 | 5 | 0.1 |
| ²²⁴Ra-CaCO₃- | 3 | 11 | 10 | 5 | 0.2 |
| MP washed | 3 | 11 | 10 | 5 | 0.4 |
| with SHMP | | | | | |
| Inclusion | 3 | 10-13 | 39 | 4 | 0.08 |
| labeled ²²⁴Ra- | | | | | |
| CaCO₃-MP in | | | | | |
| EDTMP | | | | | |

MP: Microparticles

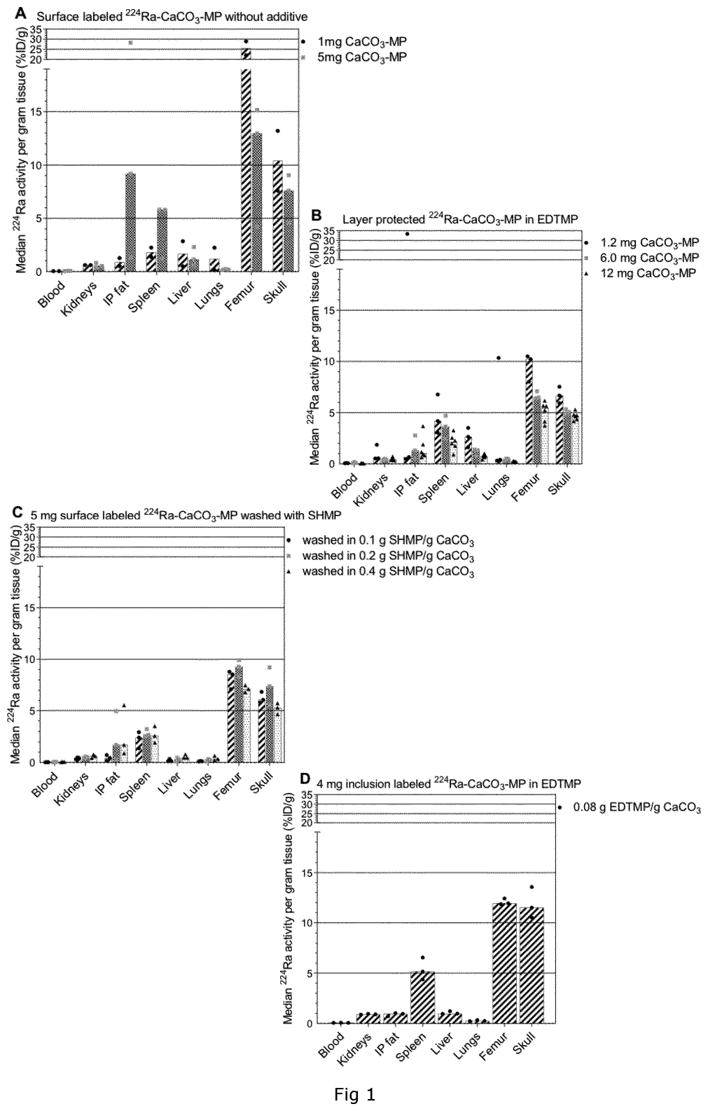

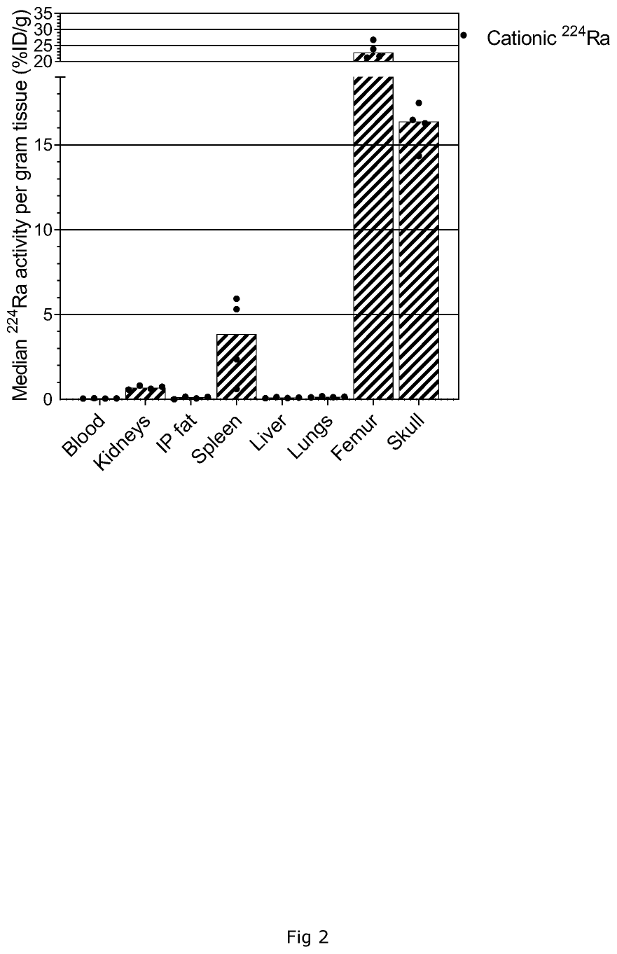

The ex vivo biodistribution data shows that the tissue distribution was largely similar between the ²²⁴Ra—CaCO₃-microparticles with (FIGS. 1B, C and D) and without (FIG. 1A) the use of a size controlling additive. All variants of the ²²⁴Ra—CaCO₃-microparticles where a phosphorus containing additive is used, have an increased IP retention of radioactivity, as reflected in the reduced uptake in femur and skull, when compared to IP administered cationic ²²⁴Ra (FIG. 2). For the variant without an additive (FIG. 1A), the uptake of ²²⁴Ra in bones after injection of 1 mg was comparable to that after IP injection of cationic ²²⁴Ra (FIG. 2), whereas the IP retention of ²²⁴Ra at 1 mg of the layer protected is significantly improved (FIG. 1B).

### Example 9—Therapeutic Effect of Radiolabeled Microparticles Dispersed in Phosphorus Compounds in an Ovarian Cancer Xenograft Model in Mice

Human ovarian cancer often leads to ascites and therefore the therapeutic effect of radiolabeled microparticles dispersed in phosphorus compounds was examined in an ovarian cancer xenograft model that produces aggressive tumor cell growth established as ascites in immunodeficient mice.

Xenografts were generated by a single IP injection of ES-2 cell suspension (1×10⁶ cells in 0.2 ml RPMI) in institutionally bred, 4-5 weeks old female athymic nude Foxn^{nu} mice. Approximately one day later, mice were treated with intraperitoneal injections of different variants of ²²⁴Ra-labeled CaCO₃ microparticles (Table 5). Mice in the control group was administered 0.9% NaCl IP. Therapeutic effect was evaluated by survival time. The mice were monitored for changes in bodyweight, behavior, posture, and appearance minimum twice per week and more frequently when they displayed signs indicating disease progression. When mice reached predetermined endpoints, which included rapid body weight loss (>10% within one week), severely impaired mobility due to ascites build-up and/or cachexia, they were euthanized by cervical dislocation.

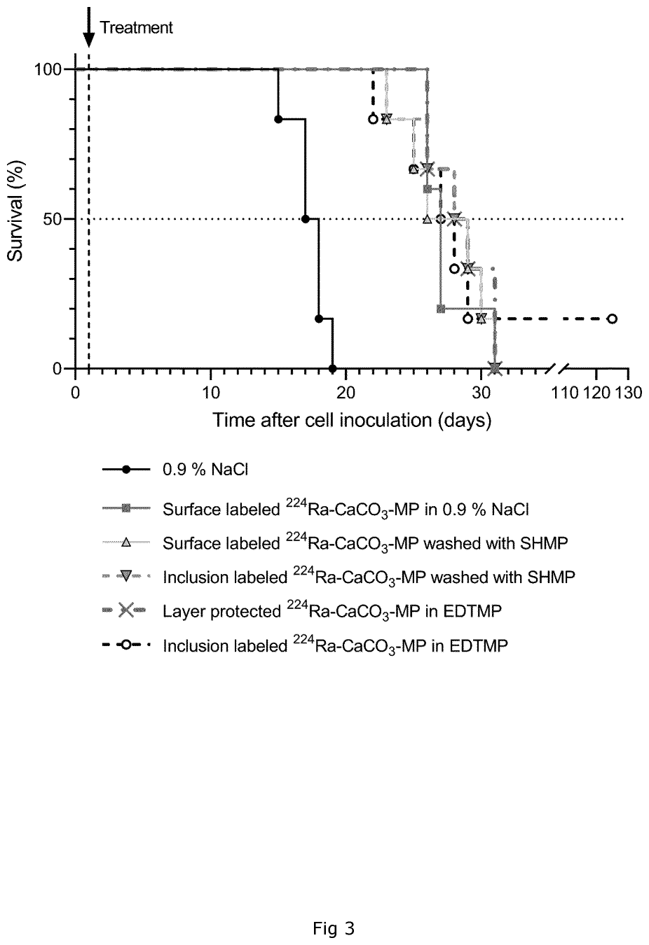

Survival times were recorded as days after tumor cell inoculation, and Kaplan-Meier survival curves are presented in FIG. 3 and median survival times in Table 6. A comparison of the survival curves revealed that all experimental groups differed significantly from the control group (pairwise log-rank tests, p≤0.0014, family-wise significance level of 0.05). At day 19 after tumor cell inoculation, all mice in the saline control group were euthanized whereas all mice in the ²²⁴Ra-labeled CaCO₃ microparticles treatment groups had not reached the study endpoint. All ²²⁴Ra-treatments had a similar extension in survival compared to the saline control, irrespective of whether phosphorus additives were used or which (EDTMP vs SHMP) or whether the microparticles were surface or inclusion labeled.

TABLE 6
Summary of selected study details and results: Efficacy of ²²⁴Ra-CaCO₃-microparticles in
human ovarian xenograft model ES-2 in immunodeficient nude mice.

| | Number | | Radioactivity | | |
|---|---|---|---|---|---|
| | of mice | Administered | based on | CaCO₃ | Median |
| | per | volume | standards¹ (s.d.) | microparticles | survival |
| Group | group | [ml/mouse] | [kBq/mouse] | [mg/mouse] | [days] |
| 0.9% NaCl | 6 | 0.40 | n/a | n/a | 17.5 |
| Surface labeled ²²⁴Ra- | 5 | 0.30 | 13.7 (0.4) | 4.1 | 27.0* |
| CaCO₃-MP in 0.9% NaCl | | | | | |
| Surface labeled ²²⁴Ra- | 6 | 0.30 | 17.9 (1.2) | 4.1 | 27.5* |
| CaCO₃-MP washed with | | | | | |
| SHMP | | | | | |
| Inclusion labeled ²²⁴Ra- | 6 | 0.45 | 15.4 (3.4) | 5.6 | 28.5* |
| CaCO₃-MP washed with | | | | | |
| SHMP | | | | | |
| Layer protected ²²⁴Ra- | 6 | 0.30 | 17.4 (2.2) | 9.0 | 28.5* |
| CaCO₃-MP in EDTMP | | | | | |
| Inclusion labeled ²²⁴Ra- | 6 | 0.35 | 20.7 (1.4) | 4.4 | 27.5* |
| CaCO₃-MP in EDTMP | | | | | |

¹Administered radioactivity per mouse is based on measurements of three standard samples per group on the Hidex Automated Gamma Counter five days post injection. The measurement of each standard was decay corrected to time of injection and the average of these are reported with the corresponding standard deviation.
*Statistically significant compared to saline control group with a family-wise significance level of 0.05. Survival curves were compared pairwise by log-rank tests and adjusted for multiple comparisons by the Bonferroni method.

### Example 10—Antitumor Efficacy of Radiolabeled Microparticles Dispersed in Phosphorus Compounds in a Syngeneic Colon Carcinoma Model in Mice

Colorectal cancer frequently results in metastases in the peritoneal cavity and hence the therapeutic effect of radiolabeled microparticles dispersed in phosphorus compounds was examined in a syngeneic model of IP colon carcinoma in immunocompetent mice.

Tumors were established by a single IP injection of the murine colorectal cancer cell line CT26.WT (5×10⁴ cells in 0.2 ml PBS) in female BALB/cAnNRj mice (Janvier Labs, France) of approximately 6 weeks age. One day later, mice were treated with intraperitoneal injections of different variants of ²²⁴Ra-labeled CaCO₃ microparticles (Table 7). Mice in the control group was administered 0.9% NaCl IP. Efficacy, as measured by survival time, was based on rapid change in body weight, development of ascites, scoring of physical appearance and development of palpable tumors in the abdomen. When mice reached these predetermined endpoints, they were euthanized by cervical dislocation.

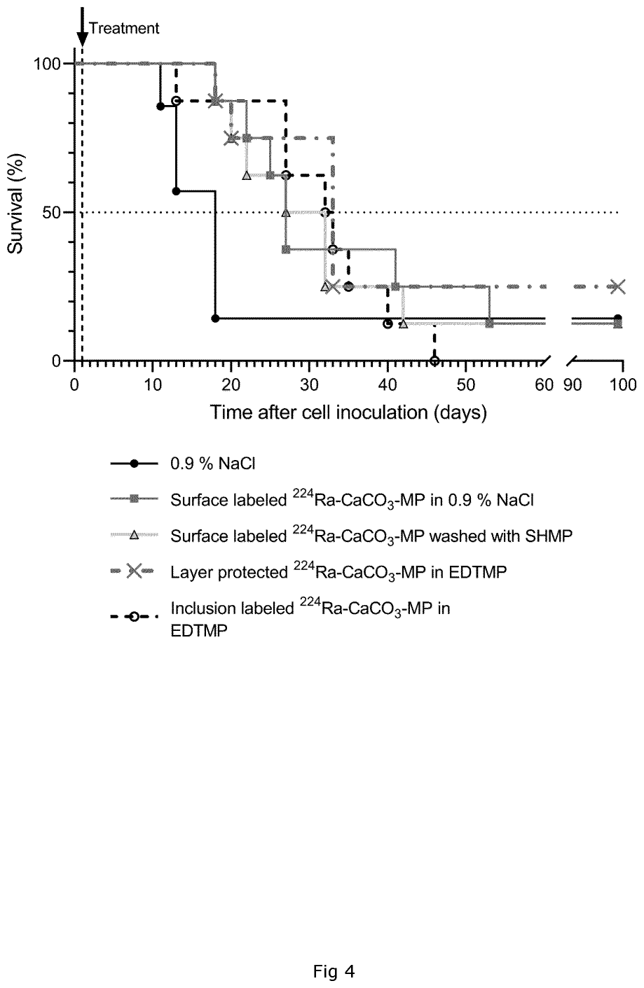

Survival times were recorded as days after tumor cell inoculation, and Kaplan-Meier survival curves are presented in FIG. 4 and median survival times in Table 7. The median survival was increased from 18 days in the control group to 27 to 33 days for the different ²²⁴Ra—CaCO₃-microparticles. All ²²⁴Ra-treatments had a similar extension in survival compared to the saline control, irrespective of whether phosphorus compounds were used or which (EDTMP vs SHMP) or whether the microparticles were surface or inclusion labeled.

TABLE 7
Summary of selected study details and results: Efficacy of intraperitoneally
administered ²²⁴Ra-labeled CaCO₃ microparticles in a syngeneic CT26.WT
colon carcinoma tumor model in BALB/c mice.

| | Number | | Radioactivity | | |
|---|---|---|---|---|---|
| | of mice | Administered | based on | CaCO₃ | Median |
| | per | volume | standards¹ (s.d.) | microparticles | survival |
| Group | group | [ml/mouse] | [kBq/mouse] | [mg/mouse] | [days] |
| 0.9% NaCl | 7 | 0.40 | n/a | n/a | 18.0* |
| Surface labeled ²²⁴Ra- | 8 | 0.40 | 26.4 (2.5) | 5.0 | 27.0 |
| CaCO₃-MP in 0.9% NaCl | | | | | |
| Surface labeled ²²⁴Ra- | 8 | 0.40 | 25.8 (4.4) | 5.0 | 29.5 |
| CaCO₃-MP washed with | | | | | |
| SHMP | | | | | |
| Layer protected ²²⁴Ra- | 8 | 0.45 | 21.5 (4.1) | 13.5 | 33.0 |
| CaCO₃-MP in EDTMP | | | | | |
| Inclusion labeled ²²⁴Ra- | 8 | 0.30 | 24.8 (2.9) | 3.8 | 32.5 |
| CaCO₃-MP in EDTMP | | | | | |

¹Administered radioactivity per mouse is based on measurements of three standard samples per group on a Wizard 2 gamma counter three days post injection. The measurement of each standard was decay corrected to time of injection and the average of these are reported with the corresponding standard deviation.

### Example 11—Lead-212 Labeled Calcium Carbonate Microparticles in Pamidronate

Calcium carbonate microparticles and ²¹²Pb solution were produced as described earlier in Examples 1 and 3. Dry CaCO₃ microparticles were suspended in WFI, ultrasonicated and washed a total of three times with WFI. Microparticles were finally suspended in 0.9% NaCl at a concentration of 25-50 mg/ml, and pamidronate disodium was added to the suspension for a relative pamidronate/CaCO₃ concentration of 0.01 g/g. In a sealed headspace vial, the suspension was autoclaved at 121° C. for 20 min before being cooled down to room temperature. Lead-212 solution was pH neutralized by adding 5M ammonium acetate and 1M sodium hydroxide, 10 v/v % of each. In the radiolabeling process, a volume of ²¹²Pb solution corresponding to approximately 50% of the microparticle suspension was added to the sealed container with microparticle suspension by use of a syringe, after which the suspension was vortexed and placed on a horizontal shaker for 3 to 60 min. The activity per microparticle mass varied from 3-99 kBq/mg. In some cases, the microparticle suspension was further diluted with 0.9% NaCl after labeling procedure.

Yield of the radiolabeling was evaluated by separating the liquid phase from the microparticles in a small sample aliquot of suspension (supernatant S and particle pellet P). The radioactivity was measured on both fractions by use of either a HPGe detector or a Hidex automated gamma counter. The yield of the radiolabeling was evaluated as CPM (P)/CPM(S)+CPM(P), where CPM denotes counts per minute.

In Table 8, an overview of the yield of the radiolabeling is given. Incubation time during horizontal shaking does not influence the radiolabeling yield, but there is a trend of improved yield with higher activity concentration.

TABLE 8
Yield of ²¹²Pb labeling of CaCO₃ microparticles added
pamidronate for size control.

| CaCO₃ | ²¹²Pb activity | | |
|---|---|---|---|
| microparticle | to CaCO₃ | Duration of | Yield of |
| concentration | mass ratio | radiolabeling | radiolabeling |
| 18 mg/ml | 3 kBq/mg | 3 min | 94% ⁽¹⁾ |
| 18 mg/ml | 3 kBq/mg | 30 min | 94% ⁽¹⁾ |
| 18 mg/ml | 3 kBq/mg | 60 min | 94% ⁽¹⁾ |
| 25 mg/ml | 41 kBq/mg | 3 min | 99% |
| 25 mg/ml | 99 kBq/mg | 3 min | 100% |

⁽¹⁾ Average from two parallel radiolabeled samples.

### Example 12—Size Control of Radiolabeled Calcium Carbonate Microparticles with and without Addition of a Calcium Carbonate Layer by Addition EDTMP

The size of unlabeled, mock labeled, and radiolabeled CaCO₃-MPs (MPs: microparticles) in suspension with varying concentration of EDTMP was measured with laser diffraction (Mastersizer 3000, Malvern Instruments Ltd., Worcestershire, UK). The unautoclaved CaCO₃-MPs used as raw material for radio- and mock labeling were used as reference, by dispersing a small amount of dried CaCO₃-MPs in water and ultrasonicating to disperse. Size stability over time was evaluated in radiolabeled CaCO₃-MPs by measuring after seven days of storage at room temperature; surface labeled MPs were compared with layer encapsulated MPs.

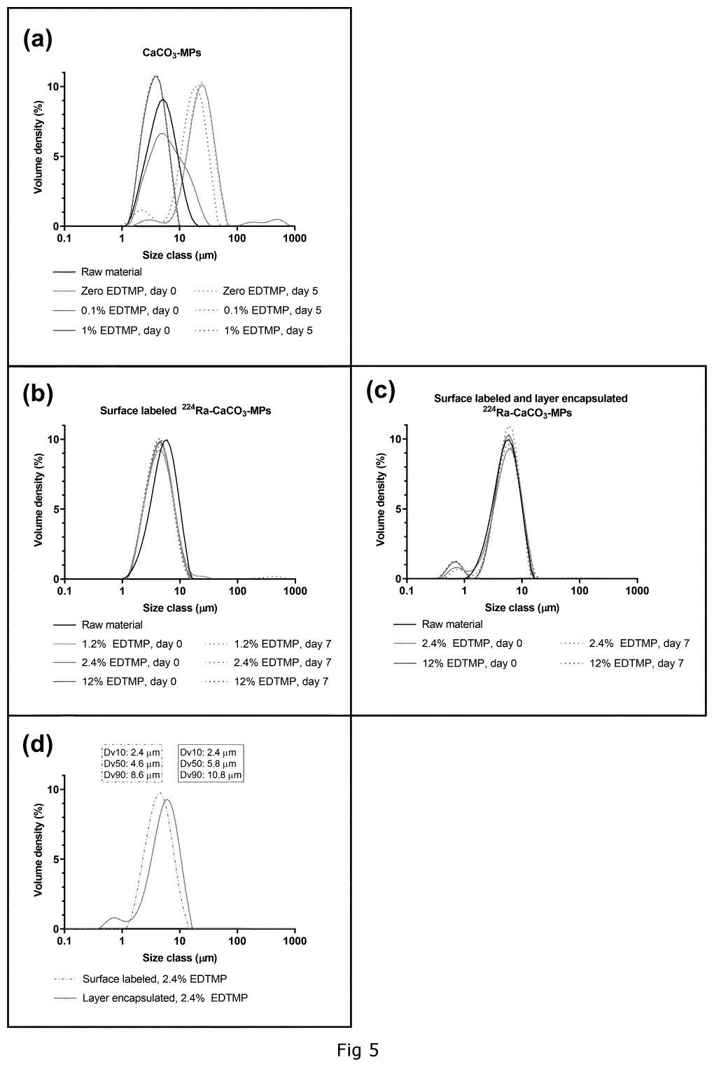

The ability of EDTMP to control size of calcium carbonate microparticles that have been radiolabeled with radium-224 and further encapsulation by addition of a layer of calcium carbonate before sterilization of the suspension by autoclaving was examined. FIG. 5 show that addition of at least 1% EDTMP resulted in a size control of the microparticles

This example shows that size control by EDTMP also is achieved for a radioalabeled, and layer encapsulated product.

### Example 13—Size Control of Calcium Carbonate Microparticles by Addition of EDTMP Slow Down Sedimentation Rate of Microparticles in Suspension

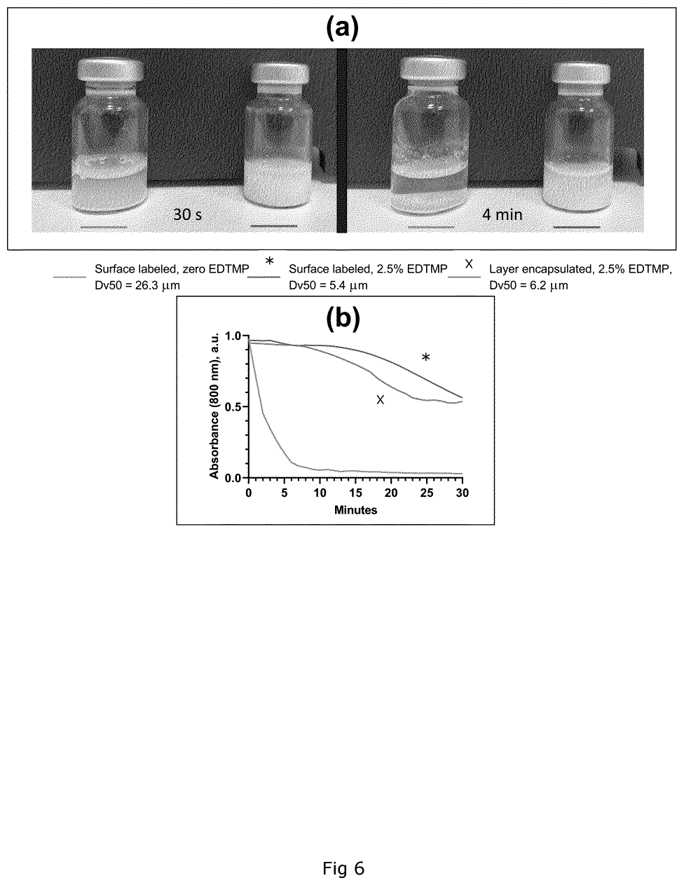

The ability of MPs to remain suspended in solution was evaluated by the sedimentation rate, which was investigated by visual inspection of samples and by evaluating the turbidity of different suspensions of non-radioactive mock labeled CaCO₃-MPs, with and without EDTMP. Turbidity was assessed by diluting the CaCO₃-MP suspension with water (water for injection), and then measuring the change in optical density at a wavelength of 800 nm over 30 min using a spectrophotometer (Hitachi U-1900, Hitachi High-Tech, Tokyo, Japan). The 800 nm wavelength was chosen to reduce potential light absorbance by CaCO₃ and improve light scattering by particles. A decrease in optical density with time is, therefore, directly related to decreased light scattering by MPs, and thereby a decreased turbidity of the sample due to sedimentation. See FIG. 6.

This example shows that the sedimentation rate is reduced by reduction of particle size. There are improved features for handling of suspension with advantage in terms of clinical administration of the product.

### Example 14—Radiochemical Properties Depending on EDTMP and Layer Encapsulation

Radiochemical purity was defined as the percentage of radionuclides retained on the MPs after a certain period. A small aliquot of suspension was separated into MP fraction P and supernatant fraction S by centrifugation. The percentage radiochemical purity, % RCP, was defined as the proportion of radioactivity in the P fraction:

CPM(P)/CPM(P+S), with CPM denoting counts per minute. The radioactivity in the two fractions was measured separately using a Hidex Automatic Gamma Counter (Hidex Oy, Turku, Finland). Radioactivity of ²¹²Pb was quantified by counts in the 60-110 keV window. For ²²⁴Ra, radioactivity was determined indirectly by assuming transient equilibrium between ²²⁴Ra and progeny ²¹²Pb after allowing the two fractions to decay for at least two days, and then measuring ²¹²Pb activity in the 65-345 keV window, in which gamma energy and X-rays mainly originated from this daughter. Sampling and measurement were repeated after up to seven days of storage at room temperature to evaluate the stability of ²¹²Pb and ²²⁴Ra % RCP over time.

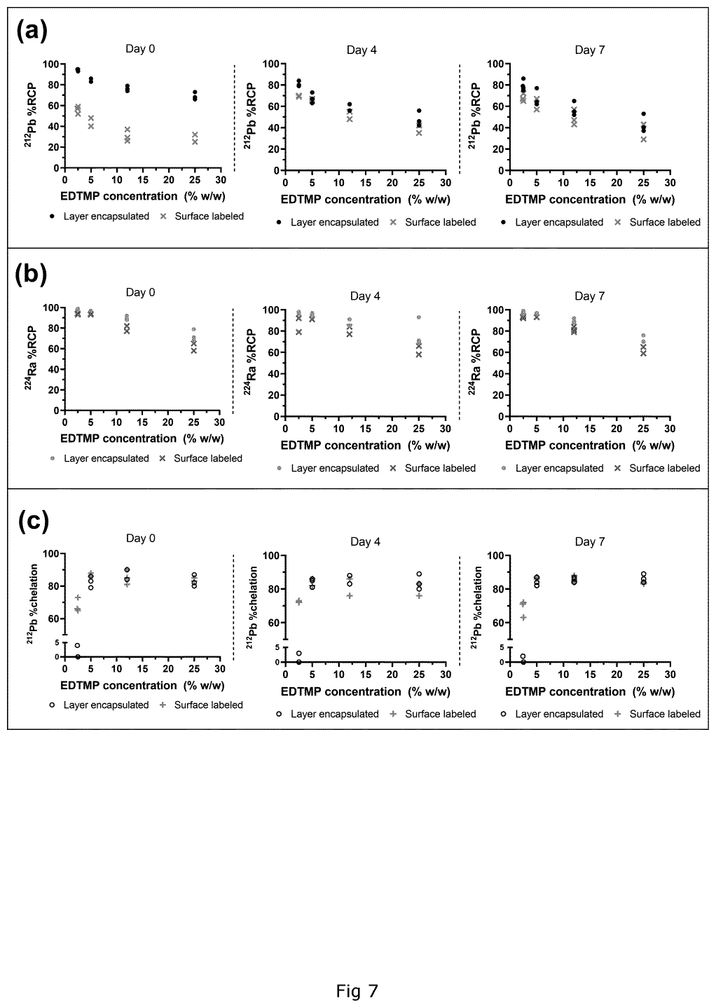

The complexation between released ²¹²Pb from MPs and EDTMP in the solution was evaluated in the liquid phase of different variants of ²²⁴Ra—CaCO₃-MPs (FIG. 7). The liquid fraction was first separated from the MPs by centrifugation. The degree of ²¹²Pb-EDTMP complexation in the obtained supernatant was then measured using instant thin layer chromatography (ITLC) strips (Tec-Control Chromatography Systems #150-772, Biodex Medical Systems, Inc., NY, United States). Chelated ²¹²Pb will migrate with the mobile phase in this system while most (>90%) unbound ²¹²Pb²⁺ will remain at the origin line, allowing for the evaluation of ²¹²Pb-EDTMP complexation. Water (pharmaceutical grade) or 0.9% NaCl was used as the mobile phase, and the strips were cut in half after the solvent front had reached the top line. Radioactivity of ²¹²Pb in the two parts was measured with a gamma counter as described earlier. The degree of chelation was defined by the proportion of migrated ²¹²Pb in the liquid fraction of ²²⁴Ra—CaCO₃-MPs and was quantified by subtracting the unspecific migration of free ²¹²Pb²⁺ in 0.9% NaCl solution without EDTMP. Equation 1 describes the percentage chelation; A ²¹²Pb-EDTMP denotes the measured activity in the supernatant of ²²⁴Ra—CaCO₃-MPs, A ²¹²Pb denotes the measured activity of free ²¹²Pb²⁺ in 0.9% NaCl solution, and m and o denote the two parts of the ITLC strip; m denotes migrated with the mobile phase and o denotes origin line.

$$\% chelation = ( \frac{{A}_{212 Pb - EDTMP , m}}{{A}_{212 Pb - EDTMP , m} + {A}_{212 Pb - EDTMP , o}} - \frac{{A}_{212 Pb , m}}{{A}_{212 Pb , m} + {A}_{212 Pb , o}} ) \times 100 \%$$

This example shows a comparison of retention of ²¹²Pb and ²²⁴Ra on CaCO₃ microparticles with or without a layer encapsulation, showing how retention of in particular ²¹²Pb is improved by addition of the layer in a product in EDTMP.

### Example 15—Biodistribution of Radium-224 and Lead-212 after i.p. Injection of Layer Encapsulated ²²⁴Ra—CaCO₃-MPs in EDTMP

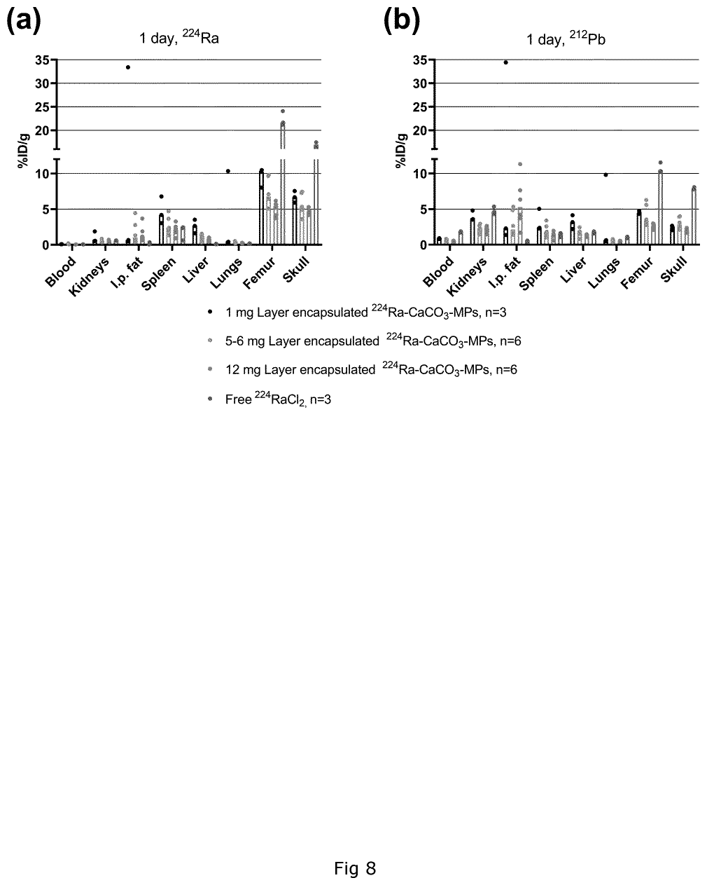

The biodistribution of layer encapsulated ²²⁴Ra—CaCO₃-MPs with added EDTMP was evaluated in institutionally bred female athymic nude mice (Hsd: Athymic Nude-Foxn1nu). Calcium carbonate microparticles were labeled and autoclaved as de-scribed earlier. The impact of mass dose (mg dose) was considered by testing doses rang-ng from 1-12 mg CaCO₃ and 6-18 kBq by creating dilutions with an isotonic infusion solution (Plasmalyte, Baxter International Inc., IL, United States). One day after a single i.p. administration, the mice were euthanized by cervical dislocation and tissue samples were obtained to measure radioactivity. Three standard samples corresponding to 25-50% of the administered dose of each treatment were used to determine the injected radioactivity dose. The radioactivity of ²¹²Pb and ²²⁴Ra of tissue and standard samples was measured using a gamma counter as described above, from which the percentage injected dose per gram tissue (% ID/g) was calculated. Correction for decay and/or ingrowth of ²²⁴Ra and ²¹²Pb was not performed in the calculation of the % ID/g for two reasons. Firstly, standard samples and tissue samples were counted with less than 2-3 h time interval (i.e., 3% of the half-life of 224Ra) and secondly, error propagation as a result of uncertainty in the measurement of ²²⁴Ra when the measured activity was close to or below the limit of quantification of the instrument could be avoided. As a reference for the skeletal accumulation of free ²²⁴Ra one day after i.p. injection, one group of mice received ˜30 kBq ²²⁴RaCl₂ prepared as described previously be Westrøm et al. See FIG. 8.

This example shows a comparison of the biodistribution of both radium-224 and lead-212 between different amount of layered encapsulated microparticles and free radium-224. Reduced levels of bone uptake with increasing amount of microparticles.

### Example 16—Distribution of 208Pb and ²¹²Pb on Layer Encapsulated Microparticles with 2.5% (w/w) EDTMP

The cumulative amount of the chemically equivalent stable daughter nuclide ²⁰⁸Pb adsorbed on the MPs increases with time, although this seems to have little effect on the adsorption of ²¹²Pb. For the surface labeled variant at this EDTMP concentration, % RCP of ²¹²Pb was only 56% on day zero, with an increase to 70% on day four.

### Example 17—Adsorption of ²¹²Pb²⁺ and ²¹²Pb-EDTMP to Calcium Carbonate Microparticles

The known complexation property of EDTMP with both ²¹²Pb and calcium indicates that it is also possible for the ²¹²Pb-EDTMP complex to associate with the MPs. To test this hypothesis, adsorption of ²¹²Pb on non-radioactive mock labeled CaCO₃-MPs, both with and without layer encapsulation, was evaluated after the addition of a solution of ²¹²Pb-EDTMP. It was found that 17-20% of the ²¹²Pb-EDTMP adsorbed on the MPs; the adsorption increased to 96% when unbound ²¹²Pb²⁺ (²¹²PbCl₂) was added instead. The reduced adsorption of ²¹²Pb-EDTMP is in line with the general observation that the % RCP of ²¹²Pb of both surface labeled MPs and layer encapsulated MPs decreased at higher EDTMP concentrations in the MP suspension.

### Example 18—Preparation of Smaller Microparticles (CaCO₃ SMPs)

Calcium carbonate microparticles has been produced by two different procedures to create particles with two distinct size populations. One procedure of CaCO₃ microparticles production was as detailed in Example 1, the other procedure was similar to the first, however, glycerol was added during the spontaneous precipitation reaction in an attempt to produce smaller microparticles (CaCO₃ SMPs). Before mixing 1 M solutions of CaCl₂) and Na₂CO₃ (Merck), glycerol (Sigma-Aldrich) was added to each solution to a concentration of 50% (v/v), diluting the solutions to 0.5 M. The two solutions were then combined under vigorous stirring with an overhead stirrer operating at 6000 RPM for 30 min. The resulting precipitate of CaCO₃ SMPs was washed three times with water for injection (WFI) before drying at 180° C. Size distribution of microparticles were measured by a laser diffraction particle size analyzer (Mastersizer 3000, Malvern Instruments Ltd, Worcestershire, UK). The CaCO₃ MP and CaCO₃ SMPs used in Example 19, 20 and 21 had volume-based median diameter of approximately 5 μm and 2 μm, respectively. Labeling with ²¹²Pb was performed as described in example 11.

### Example 19—Radiochemical Properties of Various CaCO₃ Particles in Suspension after Autoclaving and Labeling with ²¹²Pb

Measurements of the ²¹²Pb radiolabeled particles (produced as in Example 1 or Example 18 and labeled as described in Example 11) included determination of radioactivity concentration, radiochemical yield/purity, over-time-stability, and possible breakthrough of the parent nuclide ²²⁴Ra. Radioactivity level of ²¹²Pb solution and the vial of labeled CaCO₃ particles in suspension was measured using an ionization chamber dose calibrator (Capintec Inc., NJ, USA). Percentage radiolabeling yield, % RCY, was determined by separating a small aliquot of suspension into particle fraction P and supernatant fraction S to measure the portion of adsorbed ²¹²Pb on the particles. The two fractions were measured separately on an automatic gamma counter (Hidex Automatic Gamma Counter, Hidex Oy, Turku, Finland). % RCP was calculated as the ratio CPM(P)/CPM (P+S), where CPM denotes counts per minute in each fraction, to denote the fraction of adsorbed ²¹²Pb on the particles.

In vitro stability was defined as the percentage retained ²¹²Pb on microparticles in an in vitro setup consisting of incubation of ²¹²Pb—CaCO₃ MPs/SMPs in an isotonic infusion solution (Plasmalyte, Baxter, IL, USA) with pH of approximately 7 supplemented with 10 g/L human serum albumin (Sigma-Aldrich, MO, USA) in 37° C. environment for 1.5-21 h. Dilution was performed from an initial CaCO₃ concentration of 25 mg/ml down to of 2 mg/ml or 6 mg/ml. The diluted particles were separated from incubation solution and radioactivity in the two parts were measured as described above for determination of RCP.

All the variants had a ²¹²Pb % RCP of >90% in these experiments, dilution and incubation at 2-6 mg/ml resulted in release of ²¹²Pb into the incubation solution that appeared to be particle type and concentration dependent. Retention of ²¹²Pb on particles was lowest for the largest recrystallized particles and the lowest concentrations, with negligible contribution from prolonged duration of the incubation from 90 min to 21 h. The retention of ²¹²Pb on vaterite microparticles was high with a trend of higher stability for the SMPs compared to the MPs.

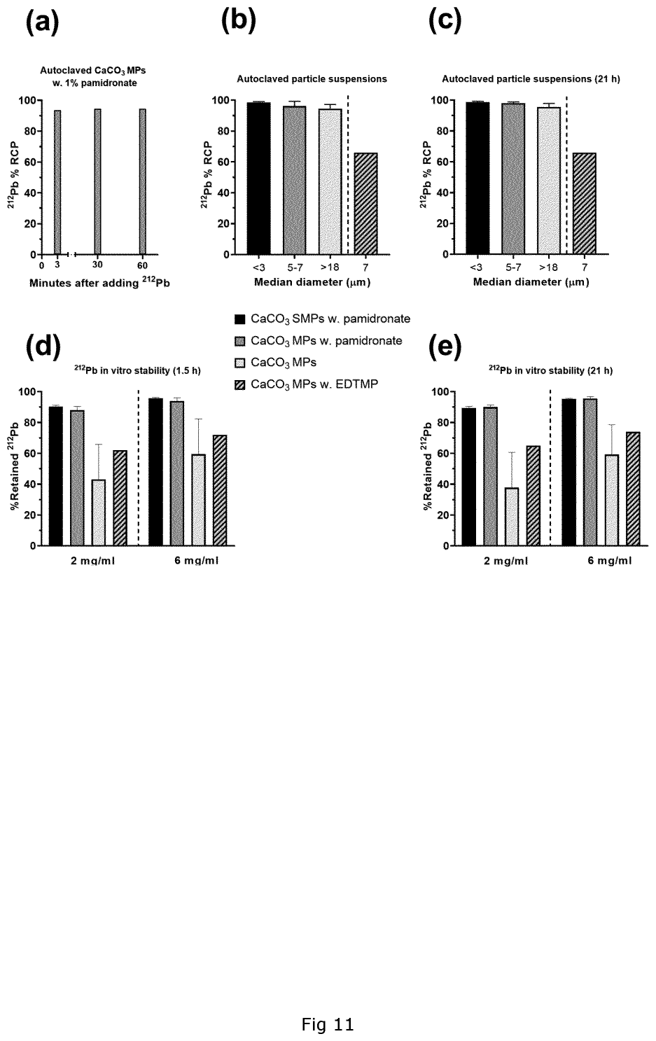

The results are shown in FIG. 11.

### Example 20—Biodistribution of Intraperitoneally Administered ²¹²Pb-Labeled CaCO₃ Microparticles in Mice

²¹²Pb—CaCO₃ MPs and ²¹²Pb—CaCO₃ SMPs was labeled as in Example 11. The biodistribution of lead-212 was evaluated after i.p. administration of ²¹²Pb—CaCO₃ MPs to tumor-free mice and compared to i.p. administration of free ²¹²Pb²⁺ in 0.9% NaCl (²¹²PbCl₂). A dose of 5 mg ²¹²Pb—CaCO₃ MPs with volume-based median particle diameter of 5 μm was investigated. Mice were sacrificed by cervical dislocation two, six, and 24 hours after the treatment and tissue samples were collected for radioactivity measurements and calculation of the percentage injected dose per gram tissue (% ID/g). In a second experiment, the biodistribution of 5 mg ²¹²Pb—CaCO₃ MPs and ²¹²Pb—CaCO₃ SMPs with volume-based median diameter below 3 μm were compared. Radioactivity measurements were performed with a Hidex automatic gamma counter with appropriate calibration factor to obtain Bq data. The measurements were decay corrected using the 10.6 h half-life of ²¹²Pb. Re-measurement of kidney and liver samples was performed after >24 h of decay to ensure transient equilibrium between ²¹²Pb and daughters. High levels of ²¹²Pb could be detected in the kidneys two hours after ²¹²PbCl₂ was injected and with considerable amounts in the blood, liver, and skeleton. The % ID/g was significantly reduced in these tissues as well as in the spleen for the MP-bound ²¹²Pb. After 6 h, the levels of ²¹²Pb in the kidneys were reduced for both variants, indicating clearance, but remained stable in the skeleton and with significantly higher % ID/g for MP-bound ²¹²Pb compared to ²¹²PbCl₂ in these tissues. After 24 h, i.e., more than two physical half-lives of ²¹²Pb, the % ID/g in most of the soft tissues was reduced and no statistical difference between the MP-bound and free variant was detected apart from in the skull (p<0.020), and the ²¹²Pb accumulated in the skeleton had increased with respect to the earlier timepoints for the MPs.

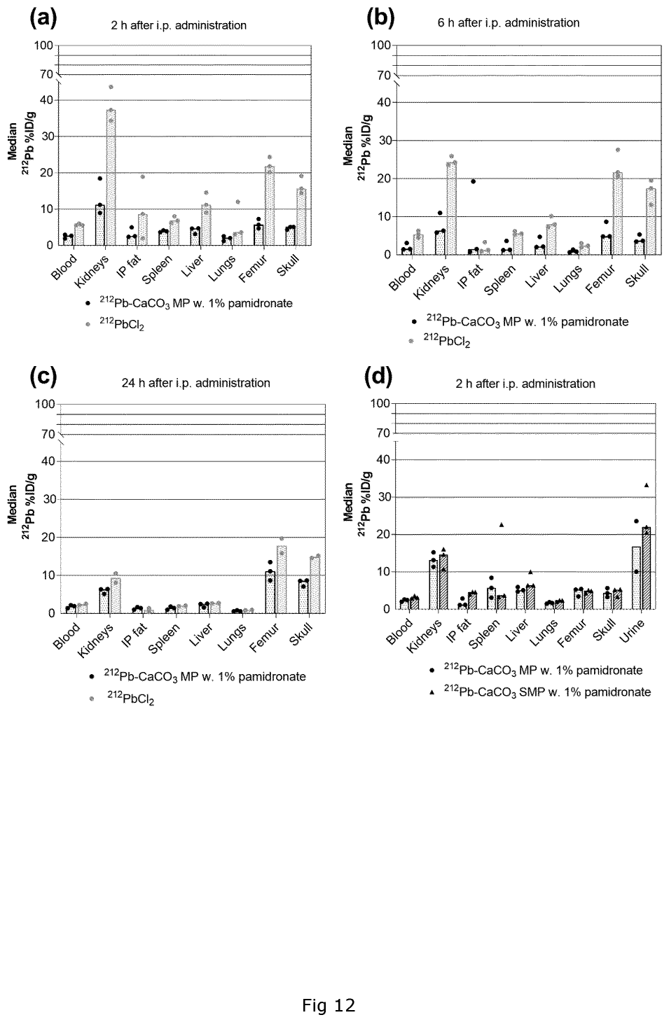

The results are shown in FIG. 12.

### Example 21—Therapeutic Efficacy of Intraperitoneally Administered ²¹²Pb-Labeled CaCO₃ Microparticles in Mice

A study of the therapeutic efficacy of ²¹²Pb—CaCO₃ MPs 1% pamidronate for treatment of cavitary cancers was performed in an i.p. xenograft mouse model of ovarian cancer. Nude mice were inoculated i.p. with 300,000 ES-2 cells (ATCC, Wesel, Germany) and treated with a single i.p. dose of 2-5 mg and 63-430 kBq ²¹²Pb—CaCO₃ MPs the day after. All radioactivity doses were measured and calculated retrospectively from standard samples as described above. Control animals were given saline or non-labeled CaCO₃ MPs suspended in saline and 1% (w/w) pamidronate. Significant therapeutic effect was observed for all of the tested doses of ²¹²Pb—CaCO₃ MPs in pamidronate compared to the saline control and non-labeled CaCO₃ MPs with pamidronate. The effect appeared dose dependent for the labeled MPs.

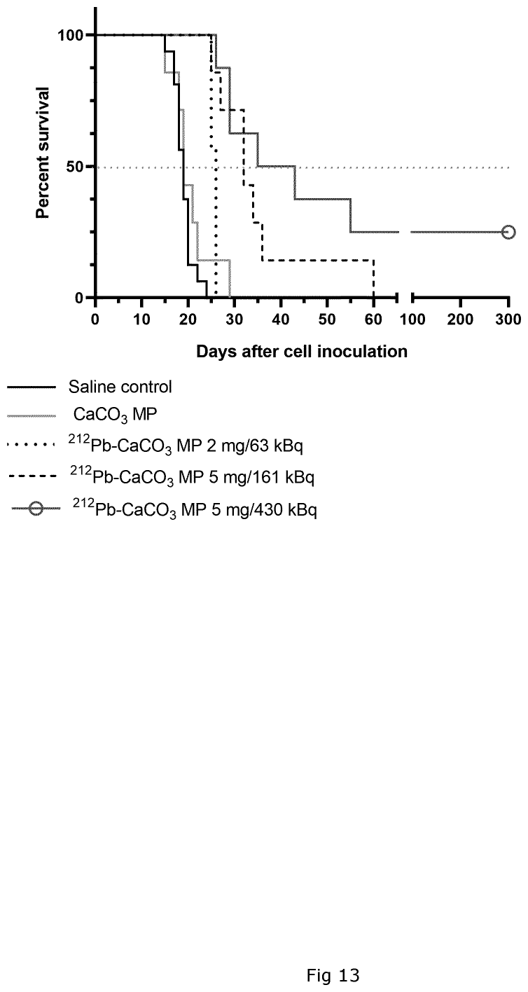

The results are shown in FIG. 13.

### Items

1. A particle comprising a degradable compound, a radionuclide, and a phosphorus containing additive.
2. The particle according to item 1, wherein the degradable compound is selected from the group consisting of CaCO₃, MgCO₃, SrCO₃, BaCO₃, calcium phosphates including hydroxyapatite Ca₅(PO₄)₃(OH) and fluoroapatite, and composites with any of these as a major constituent.
3. The particle according to items 1-2, wherein the degradable compound is CaCO₃.
4. The particle according to items 1-3, wherein the phosphorus containing additive is a phosphate selected from the group consisting of orthophosphate, linear oligophosphates and polyphosphates, and cyclic polyphosphates.
5. The particle according to items 1-3, wherein the phosphorus containing additive is a polyphosphate selected from the group consisting of pyrophosphate, tripolyphosphate and triphosphono phosphate.
6. The particle according to items 1-3, wherein the phosphorus containing additive is a cyclic polyphosphate which is sodium hexametaphosphate (SHMP).
7. The particle according to items 1-3, wherein the phosphorus containing additive is a phosphonate.
8. The particle according to item 7, wherein the phosphonate is a bisphosphonate.
9. The particle according to item 8, wherein the bisphosphonate is selected from the group consisting of Etidronate, Clodronate, Tiludronate, Pamidronate, Neridronate, Olpadronate, Alendronate, Ibandronate, Risedronate, and Zoledronate.
10. The particle according to item 7, wherein the phosphonate is a polyphosphonate.
11. The particle according to item 10, wherein the polyphosphonate is selected from the group consisting of EDTMP-ethylenediamine tetra(methylene phosphonic acid), DOTMP-1,4,7,10-Tetraazacyclododecane-1,4,7,10-tetrayl-tetrakis(methylphosphonic acid) and DTPMP-diethylenetriaminepenta(methylene-phosphonic acid).
12. The particle according to items 1-11, wherein the radionuclide is selected from the group consisting of ²²⁵Ra, ²²⁴Ra, ²²³Ra, ²²⁵Ac, ²¹²Bi, ²²⁷Th, ²¹¹At, ²¹³Bi, ²¹²Pb, ⁶⁴Cu, ⁶⁷Cu, ¹⁶⁶Ho, ¹⁷⁷Lu ³²P, ¹⁸⁶Re, ¹⁸⁸Re, ¹⁵³Sm, ⁸⁹Sr, ¹⁶¹Tb, ⁹⁰Y, ²²⁰Rn, ²¹⁶Po, ²¹²Po, ²⁰⁸Tl, ¹⁸F, ⁶⁷Ga, ⁸⁶Y, ^{99m}Tc, ¹¹¹In, ²⁰³Pb, ⁸³Sr, ¹⁵²Tb and ¹⁵⁵Tb.
13. The particle according to items 1-12, wherein the radionuclide is selected from the group consisting of alpha-radionuclides suitable for therapy consisting of ²²⁵Ac, ²¹¹At, ²¹³Bi, ²¹²Bi, ²²⁵Ra, ²²⁴Ra, ²²³Ra and ²²⁷Th.
14. The particle according to items 1-12, wherein the radionuclide is selected from the group consisting of beta-radionuclides suitable for therapy consisting of ⁶⁴Cu, ⁶⁷Cu, ¹⁶⁶Ho, ¹⁷⁷Lu, ³²P, ¹⁸⁶Re, ¹⁸⁸Re, ¹⁵³Sm, ⁸⁹Sr, ¹⁶¹Tb, ⁹⁰Y.
15. The particle according to items 1-12, wherein the radionuclide is a beta emitter with alpha-progenies suitable for therapy which is ²¹²Pb.
16. The particle according to items 1-12, wherein the radionuclide is selected from the group consisting of alpha-emitting ²²⁴Ra with the progeny radionuclides ²²⁰Rn, ²¹⁶Po, ²¹²Pb, ²¹²Bi, ²¹²Po and ²⁰⁸Tl.
17. The particle according to items 1-12, wherein the radionuclide is selected from the group consisting of radionuclide suitable for imaging consisting of ¹⁸F, ⁶⁷Ga, ⁸⁶Y, ^{99m}Tc, ¹¹¹In, ²⁰³Pb, ⁸³Sr, ¹⁵²Tb and ¹⁵⁵Tb.
18. The particle according to any one of items 1-17, wherein the size of the particle is from 1 nm to 500 μm.
19. The particle according to any one of items 1-18, wherein the degradable compound is selected from the group consisting of PEG modified CaCO₃, protein modified CaCO₃ including mAbs and Fabs, carbohydrate modified CaCO₃, lipid modified CaCO₃, vitamin modified CaCO₃, organic compound modified CaCO₃, polymer modified CaCO₃ and/or inorganic crystal modified CaCO₃.
20. A composition comprising one or more particles according to any one of items 1-19.
21. A composition which is a pharmaceutical composition comprising one or more particles according to any one of items 1-19 and a diluent, carrier, surfactant, and/or excipient.
22. The pharmaceutical composition according to item 21 or the composition according to items 15, prepared with an amount of radionuclide that is 1 kBq to 10 GBq per dosing or with an amount of radionuclide that is 50 MBq to 100 GBq suitable for multidose industrial scale production.
23. The composition according to items 20, or pharmaceutical composition according to anyone of items 21-22, wherein the composition is a particle suspension comprising monodisperse or polydisperse particles as defined in items 1-19.
24. The composition or pharmaceutical composition according to any one of items 20-23, which is suitable for parenteral use, for instance for intravenous, intracavitary and/or intratumor injections.
25. The particle according to any one of items 1-19 or composition or pharmaceutical composition according to any one of items 20-24, for use as a medicament.
26. The particle according to any one of items 1-19 or composition or pharmaceutical composition according to any one of items 20-24, for use in intracavitary therapy, radioembolization or radiosynovectomy.
27. The particle according to any one of items 1-19 or composition or pharmaceutical composition according to any one of items 20-24, for use in the treatment of cancer.
28. The particle according to any one of items 1-19 or composition or pharmaceutical composition according to any one of items 20-24, for use according to items 26-27, wherein the cancer is selected from the group consisting of intraperitoneal cancers, intracranial cancers, pleural cancers, bladder cancers, cardiac cancers, cancers in the subarachnoid cavity, and non-cavitary targets such as melanoma, non-small-cell-lung cancer.
29. The particle according to any one of items 1-19 or composition or pharmaceutical composition according to any one of items 20-24, for use in imaging.
30. The particle according to any one of items 1-19 or composition or pharmaceutical composition according to any one of items 20-24, for use according to items 26-27, which is used in combination with other cancer therapies, such as chemotherapy like taxanes (e.g. paclitaxel, docetaxel), platins (e.g. carboplatin, cisplatin), doxorubicin, mitomycin), DNA repair inhibitors such as PARP inhibitors (e.g. Olaparib, Rucaparib, Niraparib, Talazoparib, Veliparib, Pamiparib, CEP 9722, E7016, and 3-Aminobenzamide), and radioimmunotherapies.
31. The particle according to any one of items 1-19 or composition or pharmaceutical composition according to any one of items 20-24, which is a medical device or is comprised in a medical device.
32. The composition or the pharmaceutical composition according to items 20-24, wherein the concentrations of phosphonates and or phosphate compounds are 1 microgram to 1000 milligram per ml, such as 0.1 mg to 10 mg per ml of final solution, or 1 microgram to 1000 milligram per gram particles in the final solution.
33. A method for preparing a particle according to any one of items 1-19, the method comprising bringing a degradable compound, a radionuclide, and a phosphorus containing additive in contact with each other with or without using a carrier for the radionuclide.
34. A method for preparing a particle according item 33, wherein a degradable compound and a radionuclide has formed a particle in an initial step, which subsequently is coated with the phosphorus containing additive.
35. A composition or suspension comprising a particle, wherein the particle comprises a degradable compound, a radionuclide and a phosphorus containing additive, and wherein the phosphorus containing additive is associated with the particle by being present in the composition or suspension.
36. The composition or suspension according to item 35, wherein the phosphorus containing additive is part of the particle.
37. The composition or suspension according to items 35-36, wherein phosphorus containing additive is part of the composition or suspension of particles.
38. The composition or suspension according to items 35-37, wherein phosphorus containing additive is part of the particle and also part of the composition or suspension of particles.
39. The composition or suspension according to items 35-38, wherein the particle suspension which is a mixture of a solid phase and a liquid phase.
40. The composition or suspension according to items 35-39, wherein the phosphorus containing additive is in the liquid phase.
41. The composition or suspension according to items 35-40, wherein the phosphorus containing additive is in the solid phase.
42. The composition or suspension according to items 35-41, wherein the phosphorus containing additive is in the solid and the liquid phases.
43. The composition or suspension according to items 35-42, wherein the phosphorus containing additive is on the surface or embedded in the particles or both on the surface or embedded in the solid phase.

1. A particle comprising a degradable compound, a radionuclide, and a phosphorus containing additive wherein,
said degradable compound comprises CaCO₃ and
said phosphorus containing additive is selected from the group consisting of orthophosphates, linear oligophosphates, linear polyphosphates, cyclic polyphosphates, phosphonates, bisphosphonates and polyphosphonates.

2-21. (canceled)

22. The particle according to claim 1, wherein the phosphorus containing additive is sodium hexametaphosphate (SHMP), EDTMP-ethylenediamine tetra(methylene phosphonic acid) and/or Pamidronate.

23. The particle according to claim 1, wherein the phosphorus containing additive is EDTMP-ethylenediamine tetra(methylene phosphonic acid).

24. The particle according to claim 1, wherein the radionuclide is selected from the group consisting of ²²⁵Ra, ²²⁴Ra, ²²³Ra, ²²⁵Ac, ²¹²Bi, ²²⁷Th, ²¹¹At, ²¹³Bi, ²¹²Pb, ⁶⁴Cu, ⁶⁷Cu, ¹⁶⁶Ho, ¹⁷⁷Lu, ³²P, ¹⁸⁶Re, ¹⁸⁸Re, ¹⁵³Sm, ⁸⁹Sr, ¹⁶¹Tb, ⁹⁰Y, ²²⁰Rn, ²¹⁶Po, ²¹²Po, ²⁰⁸Tl, ¹⁸F, ⁶⁷Ga, ⁸⁶Y, ^{99m}Tc, ¹¹¹In, ²⁰³Pb, ⁸³Sr, ¹⁵²Tb and ¹⁵⁵Tb.

25. The particle according to claim 1, wherein the radionuclide is a beta emitter with alpha-progenies suitable for therapy which is ²¹²Pb with progeny radionuclides ²¹²Bi, ²¹²Po and ²⁰⁸Tl.

26. The particle according to claim 1, wherein the radionuclide is selected from the group consisting of alpha-emitting ²²⁴Ra with the progeny radionuclides ²²⁰Rn, ²¹⁶Po, ²¹²Pb, ²¹²Bi, ²¹²Po and ²⁰⁸Tl.

27. The particle according to claim 1, wherein the radionuclide is ²²⁴Ra, and the phosphorus containing additive is EDTMP-ethylenediamine tetra(methylene phosphonic acid).

28. The particle according to claim 1, wherein the radionuclide is ²¹²Pb, and the phosphorus containing additive is Pamidronate.

29. The particle according to claim 1, wherein the size of the particle is from 1 nm to 500 μm.

30. A composition comprising one or more particles according to claim 1.

31. A composition, according to claim 30, which is a pharmaceutical composition further comprising a diluent, carrier, surfactant, and/or excipient.

32. The composition according to claim 30, wherein the phosphorus containing additive is associated with the particle by being present in the composition or suspension, by being part of the particle, being on the surface of the particle, being in the dispersion of the particle, being part of the composition or suspension and/or dispersion of particles, or being part of the particle and as part of the composition or suspension of particles.

33. The composition according to claim 30, prepared with an amount of radionuclide that is 1 kBq to 10 GBq per dosing or with an amount of radionuclide that is 50 MBq to 100 GBq suitable for multidose industrial scale production.

34. The composition according to claim 30, wherein the composition is a particle suspension comprising monodisperse or polydisperse particles which comprise a degradable compound, a radionuclide, and a phosphorus containing additive wherein,
said degradable compound comprises CaCO₃ and
said phosphorus containing additive is selected from the group consisting of orthophosphates, linear oligophosphates, linear and polyphosphates, cyclic polyphosphates, phosphonates, bisphosphonates and polyphosphonates.

35. The composition according to claim 30, which is suitable for parenteral use or intratumor injection.

36. A method of inhibiting a cancer comprising administering the particle of claim 1 to a subject that has a cancer, wherein the cancer is selected from the group consisting of intraperitoneal cancers, intracranial cancers, pleural cancers, bladder cancers, cardiac cancers, cancers in the subarachnoid cavity, and non-cavitary cancers.

37. The method of claim 36, further comprising administering to said subject a chemotherapy, a DNA repair inhibitor or a radioimmunotherapy.

38. The method of claim 36, wherein the concentrations of phosphonates and or phosphate compounds are 1 microgram to 1000 milligram per ml particles in the final solution.

39. A method for preparing a particle according to claim 1, the method comprising bringing a degradable compound, a radionuclide, and a phosphorus containing additive in contact with each other with or without using a carrier for the radionuclide.

40. A method for preparing a composition according claim 30, wherein a degradable compound and a radionuclide has formed a particle in an initial step, which subsequently is coated with the phosphorus containing additive or has the with the phosphorus containing additive at least partly associated with the particle.
